(19) United States
(12) Patent Application Publication
(10) Pub. No.: US 2009/0023597 A1
(43) Pub. Date: Jan. 22, 2009

(54) Single Nucleotide Polymorphism Detection from Unamplified Genomic DNA

(75) Inventors: Carrie Li Wong, San Francisco, CA (US); John J. Quinn, Madison, CT (US); Brian D. Warner, Martinez, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/282,078

(22) PCT Filed: Apr. 12, 2007
(86) PCT No.: PCT/US07/09166

§ 371 Date: Sep. 8, 2008

Related U.S. Application Data
(60) Provisional application No. 60/791,281, filed on Apr. 12, 2006.

(51) Int. Cl. C40B 30/04; C12Q 1/68

(52) U.S. Cl. 506/9; 435/6

(57) ABSTRACT

The present invention provides methods, compositions and systems for the specific and selective detection of multiple single nucleotide polymorphisms (SNPs) from genomic DNA. Importantly, the inventive systems and methods eliminate the need for costly, time- and labor-intensive gene amplification that is generally carried out prior to SNP detection. Also provided are kits useful to perform the inventive methods.

### RELATED APPLICATIONS

This application claims priority from Provisional Application U.S. Ser. No. 60/791,281 filed on Apr. 12, 2006 and entitled “Single Nucleotide Polymorphism Detection from Unamplified Genomic DNA”. The provisional application is incorporated herein by reference in its entirety.

### BACKGROUND OF THE INVENTION

The ability to detect variations in nucleic acid sequences is of great importance to human genetic research and medical genetics. Genetic variation explains some of the differences among individuals, such as eye color and blood group. Genetic variation is also thought to confer susceptibility to disease (A. E. Guttmacher and F. S. Collins, New Engl. J. Med., 2002, 347: 1512-1520) and environmental toxins, and to determine individual response to pharmaceuticals (K. A. Phillips et al., JAMA, 2001, 286: 2270-2279). While multiple types of DNA variations exist, single nucleotide polymorphisms (SNPs) are the most frequent form of mutation in the human genome. SNPs, in which two or more alternative bases can occur at a given nucleotide position, are estimated to be present approximately every 1000 to 2000 bases (R. Sachidanandam et al., Nature, 2001, 409: 928-933; J. C. Venter et al., Science, 2001, 291: 1304-1351).

Because of their high density, SNPs are viewed as invaluable tools for the mapping of genes implicated in complex human diseases, drug response, and drug metabolism (A. J. Schafer and J. R. Hawkins, Nature Biotechnol., 1998, 16: 33-39; L. P. Zhao et al., Am. J. Hum. Genet., 1998, 63: 225-240; W. E. Evans and M. V. Relling, Science, 1999, 286: 487-491; L. Kryglyak, Nature Genet., 1999, 22: 139-144; J. J. McCarthy and R. Hilfiker, Nature Genet., 2000, 18: 505-508; A. D. Roses, Nature, 2000, 405: 857-865; J. J. Mc Carthy and R. Hilfiker, Nature Biotechnol., 2000, 18: 505-508; K. Lindblad-Toh et al., Nature Genet., 2000, 24: 381-386; D. E. Reich et al., Nature, 2001, 411: 199-204; J. C. Stephens et al., Science, 2001, 293: 489-493; A.-C. Syvänen, Nat. Rev. Genet., 2001, 2: 930-942; P. Y. Kowk, Annu. Rev. Genomics Hum. Genet., 2001, 2: 235-258). Detection and analysis of SNPs has therefore the potential to improve healthcare, by predicting susceptibility to disease, guiding choice of therapy, and identifying new targets. Furthermore, since SNPs are genetically stable, they can be used as genetic markers for paternity testing and for forensic identification of individuals (P. Gill, Int. J. Legal Med., 2001, 114: 204-210; A. C. Syvänen et al., Am. J. Hum. Genet., 1993, 52: 46-59) as well as in population genetics and evolutionary studies (J. G. Hacia et al., Nature Genetics, 1999, 22: 164-167; L. B. Jorde et al., Am. J. Hum. Genet., 2000, 66: 979-988; M. K. Kuhner et al., Genetics, 2000, 156: 439-447).

A number of methods have been developed for the detection of known SPNs. The most common SNP typing chemistries include hybridization-based approaches (J. G. Hacia, Nature Genet., 1999, 21: 42-47), allele-specific polymerase chain reaction (R. K. Saiki et al., Proc. Natl. Acad. Sci. USA, 1989, 86: 6230-6234; W. M. Howell et al., Nature Biotechnol., 1999, 17: 87-88), primer extension (A. C. Syvanen et al., Genomics, 1990, 8: 684-692; A. C. Synaiven, Hum. Mutat., 1999, 13: 1-10; T. Pastinen et al., Genome Res., 1997, 7: 606-614), oligonucleotide ligation (U. Landegren et al., Science, 1988, 24: 1077-1080; H. Baron et al., Nature Biotechnol., 1996, 14: 1279-1282) and enzyme-based methods such as restriction fragment length polymorphism and flap endonuclease digestion (V. Lyamichev et al., Nature Biotechnol., 1999, 17: 292-296; C. A. Mein et al., Genome Res., 2000, 10: 330-343).

A variety of technologies have been used to detect/analyze the reaction products of these SNP typing chemistries including DNA sequencing (N. M. Makridakis and J. K. Reichardt, Biotechniques, 2001, 31: 1374-1380; M. H. Shapero et al., Genome Res., 2001, 11: 1926-1934); gel or capillary electrophoresis, such as in single-stranded conformational polymorphism analysis (M. Orita et al., Proc. Natl. Acad. Sci. USA, 1989, 86: 2766-2770; P. Kozlowski and W. J. Kryzosiak, Nucleic Acids Res., 2001, 29: e71); fluorescence polarization (N. J. Gibson et al., Clin. Chem., 1997, 43: 1336-1341; X. N. Chen et al., Genome Res., 1999, 9: 492-498); high-density oligonucleotide arrays (T. Pastinen et al., Genome Res., 1997, 7: 606-614; A.-C. Syvanen, Human Mutat., 1999, 13: 1-10; M. Chee et al., Science, 1996, 274: 610-614; D. G. Wang et al., Science, 1998, 280: 1077-1082; E. A. Winzeler et al., Science, 1998, 281: 1194-1197; J. B. Fan et al., Nature Biotech., 2000, 10: 853-860; T. Pastinen et al., Genome Res., 2000, 10: 1031-1042); fluorescent bead-based technologies (M. A. Iannone et al., Cytometry, 2000, 39: 131-140; X. Hu et al., Nucleic Acids Res., 2003, 31: e43); mass spectrometry (D. J. Fu et al., Nature Biotechnology, 1998, 16: 381-384; P. Ross et al., Nature Biotechnol., 1998, 16: 1347-1351; K. Tang et al., Proc. Natl. Acad. Sci., 1999, 96: 10016-10020; T. J. Griffin and L. M. Smith, Trends Biotechnol., 2000, 18: 77-84) including MALDI-TOF spectrometry (J. Stoerker et al., Nature Biotechnol., 2000, 18: 1213-1216; S. M. S. Bray et al., Hum. Mutat., 2001, 17: 296-304); pyrosequencing (A. Ahmadian et al., Anal. Biochem., 2000, 280: 103-110; A. Alderbom et al., Genome Res., 2000, 10: 1249-1258; H. Fakrai-Rad et al., Hum. Mutat., 2002, 19: 479-485); and flow cytometry (H. Cai et al., Genomics, 2000, 66: 135-143; J. Chen et al., Genome Res., 2000, 10: 549-557). In addition, approaches using fluorescence resonance energy transfer have been shown to be useful for the detection of SNPs including 5′ nuclease TaqMan (P. M. Holland et al., Proc. Natl. Acad. Sci., 1991, 88: 7276-7280; K. L. Livak et al., Nature Genet., 1995, 9: 341-342); Scorpion primers (D. Whitcombe et al., Nature Biotechnol., 1999, 17: 804-807); and Molecular Beacons (S. Tyagi and F. R. Kramer, Nature Biotechnol., 1996, 14: 303-308; S. Tyagi et al., Nature Biotechnol., 1998, 16: 49-53; S. A. Marras et al., Genet. Anal., 1999, 14: 151-156).

Common to almost all SNP analysis methods currently available is an initial target amplification step using polymerase chain reaction (PCR) (R. K. Saiki et al., Science, 1988, 239: 487-491; A. Isaksson and U. Landegren, Curr. Opin. Biotechnol., 1999, 10: 11-15; M. M. Shi, Clin. Chem., 2001, 47: 164-172). Following amplification, the PCR product is either sequenced directly or probed for the SNP(s) of interest. However, PCR has significant limitations for the detection of rare variations in nucleic acid populations. In addition to being time-consuming, PCR may lead to false positive signals by amplification of contaminants and/or by base misincorporation. Thus, as evidenced by the numerous efforts directed to this effect (A. Castro and J. G. Williams, Anal. Chem., 1997, 69: 3915-3920; T. J. Griffin et al., Proc. Natl. Acad. Sci. USA, 1999, 96: 6301-6306; L. Fors et al., Pharmacogenomics, 2000, 1: 219-229; X. Qi et al., Nucleic Acids Res., 2001, 29: e116; K. V. N. Rao et al., Nucleic Acid Res., 2003, 31: e66), the development of simpler and/or more direct SNP analysis methods remains highly desirable.

### SUMMARY OF THE INVENTION

The present invention relates to improved systems and strategies for detecting single nucleotide polymorphisms (SNPs). In particular, the present invention provides compositions and methods that allow for the detection of one or more SNPs directly from genomic DNA. Compared to currently available SNP detection techniques, the inventive methods eliminate the need for costly, time- and labor-intensive gene amplification that is generally carried out prior to SNP detection. In certain embodiments, the compositions and methods of the present invention can be used in multiplex assay formats.

More specifically, in one aspect, the present invention provides a method for genotyping one or more single nucleotide polymorphic loci in a nucleic acid sample, the method comprising steps of: providing a sample comprising nucleic acid molecules of higher biological complexity relative to amplified nucleic acid molecules, the nucleic acid molecules of the sample including a plurality of target regions, each target regions having a single nucleotide polymorphic (SNP) locus; combining said sample with at least one set of primers specific for a first single nucleotide polymorphic locus in a first target region; performing primer extension to obtain extension products; and identifying the primer extension products obtained, wherein said step of identifying allows the genotype of said one or more single nucleotide polymorphic loci to be established.

In certain embodiments, at least two sets of primers are combined with the nucleic acid sample, wherein each set of primers is specific for one particular single nucleotide polymorphic locus in a particular target region.

In certain embodiments, the step of providing a sample comprising nucleic acid molecules of higher biological complexity relative to amplified nucleic acid molecules comprises steps of: obtaining a sample of genomic DNA and fragmenting the genomic DNA. For example, the sample of genomic DNA may be submitted to sonication to obtain genomic DNA fragments of less than 2 kb in size or less than 1 kb in size.

In certain embodiments, a set of primers specific for one particular single nucleotide polymorphic (SNP) locus in a particular target region comprises a first allele-specific primer and a second allele-specific primer. The first allele-specific primer comprises: (i) a 3′ portion which hybridizes to a portion of said particular target region immediately adjacent to said particular SNP locus, and has a 3′-terminal nucleotide which is complementary to a non-mutated base at said locus, and (ii) a 5′ portion which is complementary to all or part of a first pre-selected nucleic acid sequence that is different from sequences of the nucleic acid molecules of the sample. The second allele-specific primer comprises: (i) a 3′ portion which hybridizes to a portion of said particular target region immediately adjacent to said SNP locus, and has a 3′-terminal nucleotide which is complementary to a mutated base at said locus, and (ii) a 5′ portion which is complementary to all or part of a second pre-selected nucleic acid sequence that is different from sequences of the nucleic acid molecules of the sample.

In certain embodiments, a set of primers specific for a particular SNP locus in a particular target region further comprises at least one non-extendable oligonucleotide probe. The non-extendable oligonucleotide probe comprises: (i) a 5′ portion which is complementary to a portion of said particular target region, 3′ to said SNP locus and, (ii) at least two 3′-terminal nucleotides that are not complementary to said target region.

In certain embodiments, the step of performing primer extension to obtain primer extension products comprises using polymerase chain reaction (PCR). For example PCR may be conducted using a non-proofreading polymerase enzyme, such as a DNA polymerase which lacks 3′-exonuclease activity or which lacks both 3′-exonuclease activity and 5′-exonuclease activity.

In certain embodiments, the step of performing primer extension with PCR comprises extending primers in an allele-specific manner and incorporating nucleoside triphosphates from solution, a plurality of the nucleotides incorporated in the extension products being labeled nucleotides, thereby obtaining labeled primer extension products.

In such embodiments, the method may further comprise: subjecting the labeled primer extension products obtained to hybridization conditions with at least one set of pre-selected nucleic acid sequences, and determining whether hybridization occurs. In this method, each set of pre-selected nucleic acid sequences is associated with one set of primers specific for a particular SNP locus in a particular target region. Each set of pre-selected nucleic acid sequences comprises: (i) a first pre-selected nucleic acid sequence which is, at least in part, complementary to the 5′ portion of the first allele-specific of the associated primer set, and (ii) a second pre-selected nucleic acid sequence which is, at least in part, complementary to the 5′ portion of the second allele-specific primer of the associated primer set. Hybridization to the first pre-selected nucleic acid sequence indicates that the nucleic acid sample contains, at the particular SNP locus, a nucleotide which is complementary to the 3′-terminal nucleotide of the first allele-specific primer, and hybridization to the second pre-selected nucleic acid sequence indicates that the nucleic acid sample contains, at the particular SNP site, a nucleotide which is complementary to the 3′-terminal nucleotide of the second allele-specific primer.

In certain embodiments, more than one set of pre-selected nucleic acid sequences are used and each set of pre-selected nucleic acid sequences is associated with one set of primers specific for one particular single nucleotide polymorphic locus in a particular target region. The pre-selected nucleic acid sequences may be randomly generated.

In certain embodiments, the pre-selected nucleic acid sequences are immobilized on a solid support. The solid support may comprise an array. Alternatively or additionally, the solid support may comprise a set of beads.

For example, the first pre-selected nucleic acid sequence of a set of pre-selected nucleic acid sequences may be immobilized at a first pre-selected discrete location in an array and the second pre-selected nucleic acid sequence of said set may be immobilized at a second pre-selected discrete location in the array. In such an array, the first discrete location is associated with the nucleotide at the particular SNP locus being a non-mutated base, and the second discrete location is associated with the nucleotide at said locus being a mutated base.

Alternatively, the first pre-selected nucleic acid sequence of a set of pre-selected nucleic acid sequences may be immobilized on a first coded solid support and the second pre-selected nucleic acid sequence of said set may be immobilized on a second coded solid support, wherein the first coded solid support is associated with the nucleotide at the particular SNP locus being a non-mutated base, and the second coded solid support is associated with the nucleotide at said locus being a mutated base.

In certain embodiments, the step of determining whether hybridization occurs comprises a step of detecting labeled primer extension products hybridized to pre-selected nucleic acid sequences immobilized on a solid support. Detecting may be performed using a photonic, electronic, acoustic, opto-acoustic, electrochemical, electro-optic, mass-spectrometric, enzymatic, chemical, biochemical, physical technique or any combination thereof. For example, the step of detecting may be performed using a planar waveguide chip technique.

The present invention also provides kits for genotyping one or more single nucleotide polymorphic loci in a nucleic acid sample according to methods disclosed herein.

These and other objects, advantages and features of the present invention will become apparent to those of ordinary skill in the art having read the following detailed description.

### BRIEF DESCRIPTION OF THE DRAWING

FIG. 1 is a scheme showing an example of one embodiment of a SNP detection system according to the present invention. In this example, the non-extendable oligonucleotide probe is 25 bases in length. It is complementary to the same strand as the allele-specific extension primer and has 4 additional bases of non-complementary sequence at its 3′ end. Once bound to the genomic DNA, this probe can block the polymerase, which results in the formation of an extension product of a known length. The distance between the allele-specific extension primer and blocking probe determines this length.

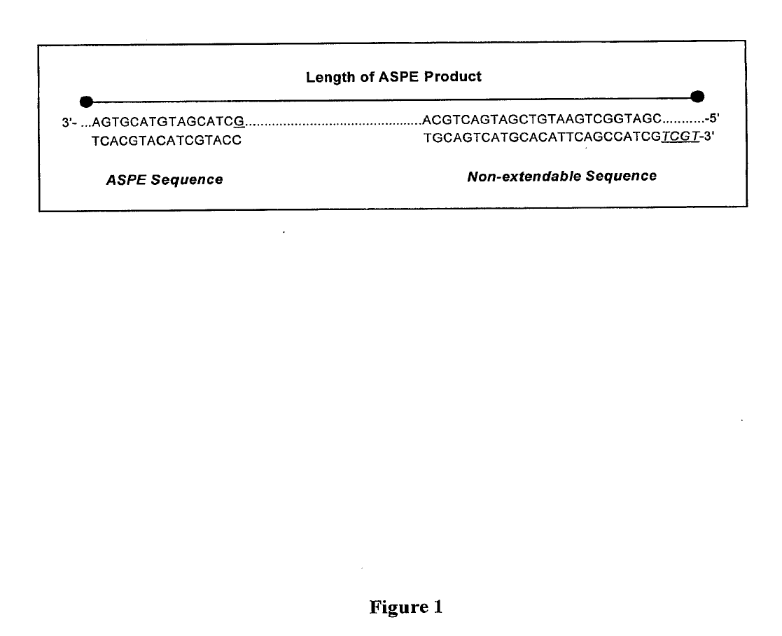

FIG. 2 is a scheme showing an example of one embodiment of a primer extension process according to the present invention. More specifically, the scheme depicts a wild type target being extended until the polymerase (lacking a 5′→3′ exonuclease activity) encounters the blocking oligonucleotide probe. Since the polymerase does not contain the 5′→3′ exonuclease activity that would hydrolyze the blocking DNA strand during extension, the extension stops.

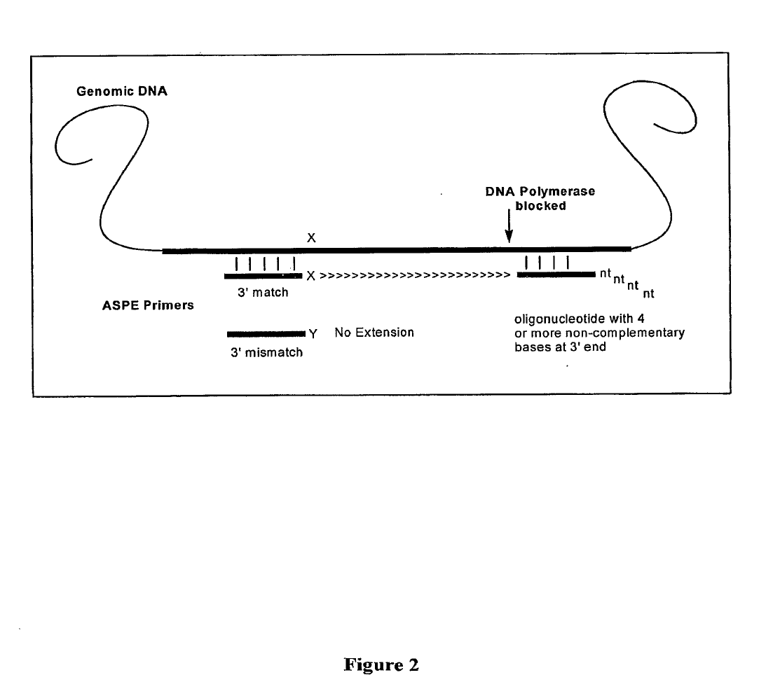

FIG. 3 shows examples of platforms suitable for the detection of multiplexed targets according to the present invention. B: biotin; WT: wild-type; VAR; Variant, Cy5: 5-N—N′-diethyl-tetramethylindodicarbocyanine.

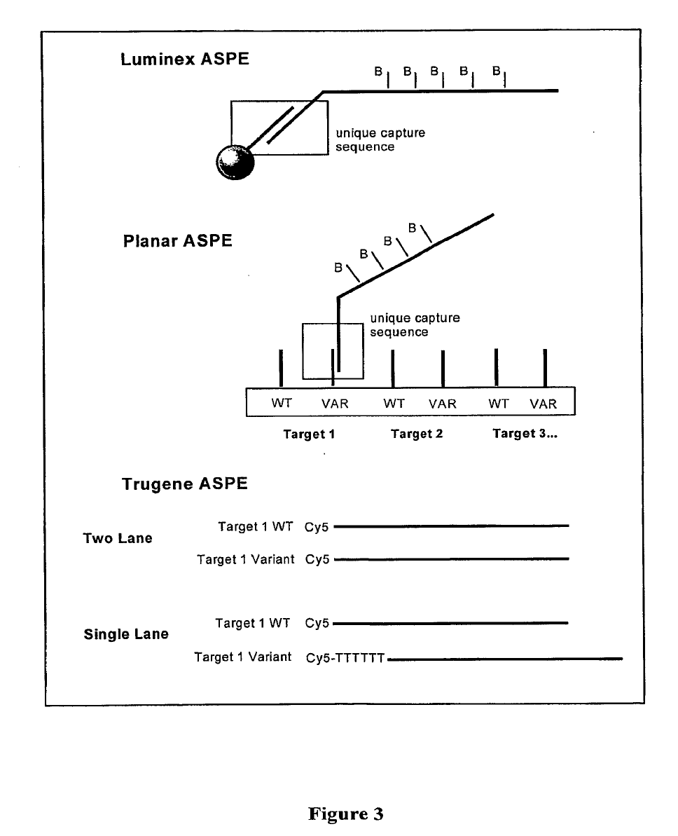

FIG. 4 is a scheme showing an example of one embodiment of an inventive method of SNP detection from genomic DNA. SA-PE: streptavidin-phycoerythrin; SA-Cy5: streptavidin-5-N—N′-diethyl-tetramethylindodicarbocyanine; PWT: Planar Waveguide Technology.

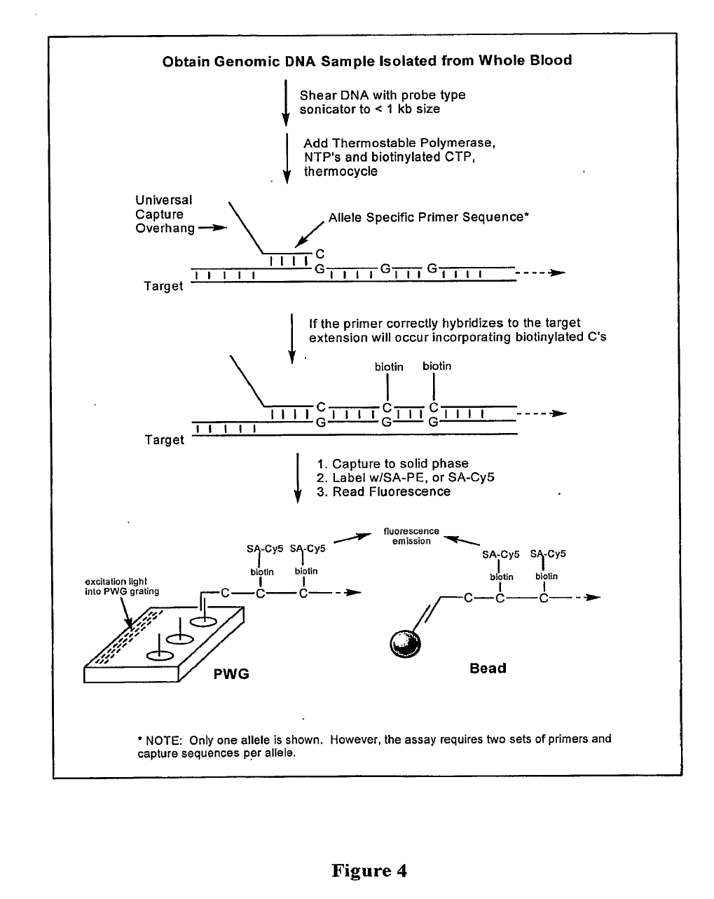

FIG. 5 is a scheme depicting the basic concept of Planar Waveguide Technology used in experiments reported in the Examples section.

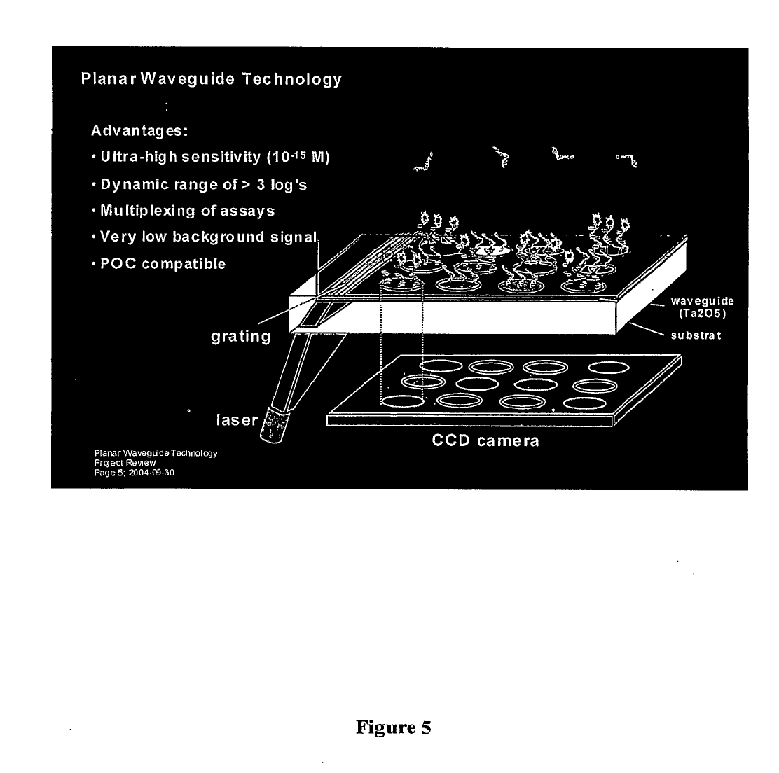

FIG. 6 is a scheme showing the workflow of inventive assays reported in the Examples section.

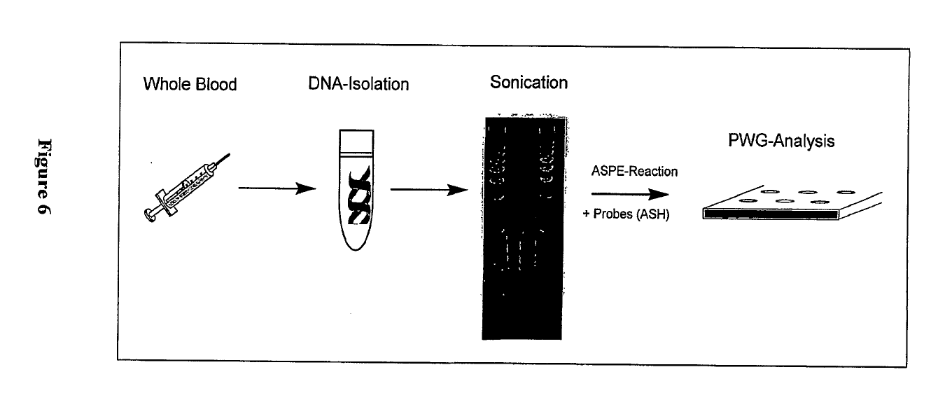

FIG. 7 is a scheme showing SNP determination using an allele-specific primer extension (ASPE) reaction according to the present invention (see the Examples section).

FIG. 8 is a scheme showing SNP determination using a ASH (allele-specific hybridization) method (see the Examples section).

FIG. 9 shows data obtained in experiments described in the Examples section using Planar Waveguide technology.

FIG. 10 is a graph showing the results of experiments carried out using an example of an inventive allele-specific primer extension (ASPE) reaction for SNPs determination and Planar Waveguide Technology for detection (see details in the Examples section).

FIG. 11 is a graph showing the results of experiments carried out using an ASH method for SNPs determination and Planar Waveguide Technology for detection, as reported in the Examples section.

### DEFINITIONS

For purposes of convenience, definitions of a variety of terms used throughout the specification are presented below.

The term “gene”, as used herein, has its art understood meaning, and refers to a part of the genome specifying a macromolecular product, be it a functional RNA molecule (such as ribosomal RNA (rRNA), transfer RNA (tRNA), etc) or a protein, and may include regulatory sequences preceding (5′ non coding sequences) and following (3′ non coding sequences) the coding sequences.

As used herein, the term “wild-type” refers to a gene, gene portion or gene product that has the characteristics of that gene, gene portion or gene product when isolated from a naturally occurring source. A wild-type gene has the sequence that is the most frequently observed in a population and is thus arbitrarily designated as the “normal” or “wild-type” sequence.

The terms “allele” and “allelic variant” are used herein interchangeably. They refer to alternative forms of a gene or a gene portion. Alleles occupy the same locus or portion on homologous chromosomes. When an individual has two identical alleles of a gene, the individual is said to be homozygous for the gene or allele. When an individual has two different alleles of a gene, the individual is said to be heterozygous for the gene. Alleles of a specific gene can differ from each other in a single nucleotide or a plurality of nucleotides, and can include substitutions, deletions and/or insertions of nucleotides with respect to each other. An allele of a gene can also be a form of a gene containing a mutation. While the terms “allele” and “allelic variant” have traditionally been applied in the context of genes, which can include a plurality of polymorphic sites, the term is also applied herein to any form of a genomic DNA sequence, which may or may not fall within a gene. Thus, each polymorphic variant of a polymorphic site is herein considered as an allele. The term “allele frequency” refers to the frequency at which a particular polymorphic variant, or allele, occurs in a population being tested (e.g., between cases and controls in an association study).

The term “polymorphism” refers to the occurrence of two or more alternative genomic DNA sequences or alleles in a population. Either of the sequences themselves, or the site at which they occur, may also be referred to as a polymorphism. A “single nucleotide polymorphism or SNP” is a polymorphism that exists at a single nucleotide position. A “polymorphic site”, “polymorphic position” or “polymorphic locus” is a location at which differences in genomic DNA exist among members of a population. While in general, the polymorphic sites of interest in the context of the present invention are single nucleotides, the term is not limited to sites that are only one nucleotide in length.

As used herein, the term “genotype” refers to the identity of an allelic variant at a particular polymorphic position in an individual. It will be appreciated that an individual's genome will contain two allelic variants for each polymorphic position (located on homologous chromosomes). The allelic variants can be the same or different. A genotype can include the identity of either or both the allelic variants. A genotype can include the identities of allelic variants at multiple different polymorphic positions, which may or may not be located within a single gene. A genotype can also refer to the identity of an allele of a gene at a particular gene locus in an individual and can include the identity of either or both alleles. The identity of the allele of a gene may include the identity of the polymorphic variants that exist at multiple polymorphic sites within the gene. The identity of an allelic variant or an allele of a gene refers to the sequence of the allelic variant or allele of a gene (e.g., the identity of the nucleotide present at a polymorphic position or the identity of the nucleotide present at each of the polymorphic positions in a gene). It will be appreciated that the identity need not be provided in terms of the sequence itself. For example, it is typical to assign identifiers such as +, −, A, a, B, b, etc, to different allelic variants or alleles for descriptive purposes. Any suitable identifier can be used. “Genotyping” an individual refers to providing the genotype of the individual with respect to one or more allelic variants or alleles.

The terms “genomic DNA” and “genomic nucleic acid” are used herein interchangeably. They refer to nucleic acid from the nucleus of one or more cells, and include nucleic acid derived from (e.g., isolated from, cloned from) genomic DNA.

The terms “sample of genomic DNA” and “sample of genomic nucleic acid” are used herein interchangeably and refer to a sample comprising DNA or nucleic acid representative of genomic DNA isolated from a natural source and in a form suitable for hybridization to another nucleic acid (e.g., as an aqueous solution). Samples of genomic DNA to be used in the practice of the present invention generally include a plurality of nucleic acid segments (or fragments) which together cover a substantially complete genome or a substantially complete portion of a genome. A sample of genomic DNA can be isolated, extracted or derived from humans, animals, plants, fungi, yeast, bacteria, viruses, tissue cultures or viral cultures, or a combination of the above. A sample of genomic DNA may be isolated, extracted or derived from solid tissues, body fluids, skeletal tissues, or individual cells. A sample of genomic DNA can be isolated, extracted or derived from fetal or embryonic cells or tissues obtained by appropriate methods, such as amniocentesis or chrorionic villus sampling.

The terms “nucleic acid”, “nucleic acid molecule”, and “polynucleotide” are used herein interchangeably. They refer to linear polymers of nucleotide monomers or analogs thereof, such as deoxyribonucleic acid (DNA) and ribonucleic acid (RNA). Unless otherwise stated, the terms encompass nucleic acid-like structures with synthetic backbones, as well as amplification products.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

The term “unamplified nucleic acid molecules” refers to nucleic acid molecules that have not been submitted to an amplification process before being analyzed, for example, before being analyzed using a SNP detection method of the present invention. Unamplified nucleic acid molecules have a higher biological complexity relative to amplified nucleic acid molecules.

The term “oligonucleotide”, as used herein, refers to a short string of nucleotides or analogs thereof. These short stretches of nucleic acid sequences may be obtained by a number of methods including, for example, chemical synthesis, restriction enzyme digestion, and PCR. As will be appreciated by one skilled in the art, the length of an oligonucleotide (i.e., the number of nucleotides) can vary widely, often depending on its intended function or use. Generally, oligonucleotides comprise between about 5 and about 150 nucleotides, usually between about 10 and about 100 nucleotides, and more usually between about 15 and about 50 nucleotides. Throughout the specification, whenever an oligonucleotide is represented by a sequence of letters (chosen from the four base letters: A, C, G, and T, which denote adenosine, cytidine, guanosine, and thymidine, respectively), the nucleotides are presented in the 5′→3′ order from the left to the right.

The term “3′” refers to a region or position in a polynucleotide or oligonucleotide 3′ (i.e., downstream) from another region or position in the same polynucleotide or oligonucleotide. The term “5” refers to a region or position in a polynucleotide or oligonucleotide 5′ (i.e., upstream) from another region or position in the same polynucleotide or oligonucleotide. The terms “3′ end” and “3′ terminus”, as used herein in reference to a nucleic acid molecule, refer to the end of the nucleic acid which contains a free hydroxyl group attached to the 3′ carbon of the terminal pentose sugar. The terms “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

The term “target nucleic acid” and “target sequence” are used herein interchangeably. They refer to a nucleic acid sequence, the presence or absence of which is desired to be determined/detected. The target sequence may be single-stranded or double-stranded. If double-stranded, the target sequence may be denatured to a single-stranded form prior to its detection. This denaturation is typically performed using heat, but may alternatively be carried out using alkali, followed by neutralization. In the context of the present invention, a target sequence comprises at least one single nucleotide polymorphic site. Preferably, target sequences comprise nucleic acid sequences to which primers can hybridize, and/or probe-hybridizing sequences with which probes (for example, non-extendable oligonucleotide probes) can form stable hybrids under desired conditions.

The term “hybridization”, as used herein, refers to the formation of complexes (also called duplexes or hybrids) between nucleotide sequences which are sufficiently complementary to form complexes via Watson-Crick base pairing or non-canonical base pairing. It will be appreciated that hybridizing sequences need not have perfect complementarity to provide stable hybrids. In many situations, stable hybrids will form where fewer than about 10% of the bases are mismatched. Accordingly, as used herein, the term “complementary” refers to a nucleic acid molecule that forms a stable duplex with its complement under assay conditions, generally where there is about 90% or greater homology. Those skilled in the art understand how to estimate and adjust the stringency of hybridization conditions such that sequences that have at least a desired level of complementarity will stably hybridize, while those having lower complementarity will not. For examples of hybridization conditions and parameters, see, for example, J. Sambrook et al., “Molecular Cloning: A Laboratory Manual”, 1989, Second Edition, Cold Spring Harbor Press: Plainview, N.Y.; F. M. Ausubel, “Current Protocols in Molecular Biology”, 1994, John Wiley & Sons: Secaucus, N.J. Complementarity between two nucleic acid molecules is said to be “complete”, “total” or “perfect” if all the nucleic acids' bases are matched, and is said to be “partial” otherwise.

The terms “probes” and “primers”, as used herein, typically refer to oligonucleotides that hybridize in a sequence specific manner to a complementary nucleic acid molecule (e.g., a nucleic acid molecule comprising a target sequence). The term “primer”, in particular, generally refers to an oligonucleotide that acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc; in an appropriate buffer solution containing any necessary co-factors and at a suitable temperature). Such a template-directed synthesis is also called “primer extension”. For example, a primer pair may be designed to amplify a region of DNA using PCR. Such a pair will include a “forward” primer and a “reverse” primer that hybridize to complementary strands of a DNA molecule and that delimit a region to be synthesized/amplified.

Typically, an oligonucleotide probe or primer will comprise a region of nucleic acid sequence that hybridizes to at least about 8, more preferably at least about 10 to about 15, typically about 20 to about 40 consecutive nucleotides of a target nucleic acid (i.e., will hybridize to a contiguous sequence of the target nucleic acid). Oligonucleotides that exhibit differential or selected binding to a polymorphic site may readily be designed by one of ordinary skill in the art. For example, an oligonucleotide that is perfectly complementary to a sequence that encompasses a polymorphic site will hybridize to a nucleic acid comprising that sequence as opposed to a nucleic acid comprising an alternate polymorphic variant.

As used herein, the term “allele-specific primer” refers to a primer whose 3′-terminal base is complementary to the corresponding template base for a particular allele at the single nucleotide polymorphic site. In certain embodiments, an allele-specific primer comprises a sequence that is perfectly complementary to a sequence of the template immediately upstream to the polymorphic site. The terms “allele-specific primer extension” and “ASPE” are used herein interchangeably. They refer to a process in which an oligonucleotide primer is annealed to a DNA template 3′ with respect to a nucleotide indicative of the presence or absence of a target allele, and then extended in the presence of labeled dNPT.

The terms “matched primer” and “mismatched primer” are used herein as an indication of the complementarity of the 3′ terminal base of the primer to the corresponding template base. A matched primer is a primer whose 3′ terminal base is complementary to the corresponding template base. Following hybridization to the template, a matched primer can be extended enzymatically. A mismatched primer is a primer whose 3′ terminal base is non-complementary to the corresponding template base. Following hybridization to the template, a mismatched primer cannot be (or cannot be significantly) extended enzymatically. It will be understood by one skilled in the art that a mismatched primer for one allele of an SNP may be a matched primer for a different allele of the SNP.

As used herein, the term “non-extendable oligonucleotide probe” refers to an oligonucleotide that is made non-extendable by adding bases to the 3′ end that are not complementary to the target sequence, and therefore do not hybridize and cannot be extended enzymatically. Other methods of making the oligonucleotide non-extendable can be used. A non-extendable oligonucleotide probe generally binds with high affinity to the template nucleic acid at a location 5′ to the termination site and effects cessation of DNA replication by DNA polymerase with respect to the template comprising the target sequence. In certain embodiments of this invention, the non-extendable oligonucleotide probe is between about 15 and about 50 nucleotides in length (e.g., between about 18 and about 30 nucleotides in length), is complementary to the same strand as the allele-specific primer, and contains a blocking sequence such as a sequence comprising at least 1, at least 2, at least 3, at least 4, or more than 4 bases of non-complementary sequence at its 3′ end. Under appropriate conditions (such as, for example, in the presence of a DNA polymerase which lacks a 5′→3′ exonuclease activity or both a 5′→3′ and 3′→5′ exonuclease activity), the non-extendable oligonucleotide probe stops the polymerase, which results in the formation of an extension product of known length.

As used herein, the term “DNA polymerase” refers to enzymes that are capable of incorporating nucleotides onto the 3′ hydroxyl terminus of a nucleic acid in a 5′ to 3′ direction thereby synthesizing a nucleic acid sequence. Examples of DNA polymerases include, but are not limited to, E. coli DNA polymerase I, the large proteolytic fragment of E. coli DNA polymerase I, commonly known as “Klenow” polymerase, “Taq” polymerase, T7 polymerase, Bst DNA polymerase, T4 polymerase, T5 polymerase, reverse transcriptase, exo-BCA polymerase, etc.

The term “nuclease activity” refers to an enzyme activity that cleaves nucleic acids at phosphodiester bonds. This activity can be either endo (i.e., the enzyme cleaves at internal phosphodiester bonds) or exo (i.e., the enzyme cleaves at the phosphodiester bond closest to either the 5′ or 3′ terminus of the nucleic acid strand). The terms “5′→3′ exonuclease activity” and “5′ exonuclease activity” are used herein interchangeably and refer to an enzyme activity that cleaves at the phosphodiester bond closest to the 5′ terminus of the nucleic acid strand. The terms “3′→5” exonuclease activity” and “3′ exonuclease activity” are used herein interchangeably and refer to an enzyme activity that cleaves at the phosphodiester bond closest to the 3′ terminus of the nucleic acid strand.

The terms “labeled” and “labeled with a detectable agent (or moiety)” are used herein interchangeably to specify that an entity (e.g., a target sequence) can be visualized, for example following hybridization to another entity (e.g., a probe). Preferably, the detectable agent or moiety is selected such that it generates a signal which can be measured and whose intensity is related to (e.g., proportional to) the amount of hybrid. Methods for labeling nucleic acid molecules are well-known in the art. Labeled nucleic acids can be prepared by incorporation of, or conjugation to, a label that is directly or indirectly detectable by spectroscopic, photochemical, biochemical, immunochemical, electrical, optical, or chemical means. Suitable detectable agents, include, but are not limited to, radionuclides, fluorophores, chemiluminescent agents, microparticles, enzymes, calorimetric labels, magnetic labels, haptens, molecular beacons, and aptamer beacons.

The term “fluorophore”, “fluorescent moiety” and “fluorescent dye” are used herein interchangeably. They refer to a molecule that absorbs a quantum of electromagnetic radiation at one wavelength, and emits one or more photons at a different, typically longer wavelength in response. Numerous fluorescent dyes of a wide variety of structures and characteristics are suitable for use in the practice of the present invention. Methods and materials for fluorescently labeling nucleic acid molecules are known in the art (see, for example, R. P. Haugland, “Molecular Probes: Handbook of Fluorescent Probes and Research Chemicals 1992-1994”, 5^{th} Ed., 1994, Molecular Probes, Inc.). Rather than being directly detectable themselves, some fluorescent dyes transfer energy to another fluorescent dye in a process of non-radiative fluorescence resonance energy transfer (FRET), and the second dye produces the detected signal. Such FRET fluorescent dye pairs are also encompassed by the term “fluorescent moiety”. The use of physically linked fluorescent reporter/quencher molecule is also within the scope of the invention. In these embodiments, when the reporter and quencher moieties are held in close proximity, such as at the ends of a nucleic acid probe, the quencher moiety prevents detection of a fluorescent signal from the reporter moiety. When the two moieties are physically separated, for example in the absence of target, the fluorescence signal from the reporter moiety becomes detectable.

### DETAILED DESCRIPTION OF CERTAIN PREFERRED EMBODIMENTS

As mentioned above, the present invention is directed to new strategies for the detection of single nucleotide polymorphisms (SNPs). The methods of the present invention do not require prior amplification of the specific sequence(s) containing the SNP(s) of interest and therefore allow for the detection of one or more SNPs directly from genomic DNA. Detection of a SNP according to the present invention relies on differential reactions occurring depending on the presence or absence of a mismatch.

### I. Inventive Oligonucleotide Primers and Probes

The methods of SNP detection disclosed herein generally include allele-specific primer extension (ASPE) and involve the use of allele-specific primers and, optionally, non-extendable oligonucleotide probes. In the inventive methods, the presence or absence of a specific SNP is detected by selective amplification, wherein one of the alleles is amplified without amplification of the other allele(s). In these methods, allele-specific primers are used that anneal to the target and whose 3′-terminal base is complementary to the corresponding template base of one allele but is a mismatch for the alternative allele(s). Since the extension starts at the 3′-end of the primer, a mismatch at or near this position has an inhibitory effect on extension (DNA polymerases extend primers with a mismatched 3′ nucleotide with a much lower efficiency than perfect matches). Therefore, under appropriate amplification conditions, only that allele which is complementary to the matched primer is amplified.

### Allele Specific Primers

Allele-specific primers for use in methods of the present invention may be any oligonucleotide that comprises an appropriate allele-specific sequence wherein the 3′-terminal nucleotide provides the desired match or mismatch for subsequent extension in the case of a match and inhibition of extension in the case of a mismatch.

More specifically, suitable allele-specific primers comprise a target nucleic acid binding domain that is of sufficient length to form stable hybrids with the template DNA under extension conditions. Generally, the target nucleic aid binding domain extends at least about 8 and less than about 100 nucleotides in the 5′ direction from the allele-specific 3′-terminal base. According to certain embodiments of the invention, allele-specific primers extend at least about 10, at least about 12, at least about 15 or at least about 20 nucleotides in the 5′ direction. According to certain embodiments of the invention, allele-specific primers extend less than about 80, less than about 60, less than about 50, less than about 40 or less than about 30 nucleotides in the 5′ direction.

In some embodiments, the target nucleic acid binding domain of an allele-specific primer is perfectly complementary to a sequence of the template immediately upstream to the single nucleotide polymorphic site. In other embodiments, the target nucleic acid domain forms a stable hybrid with the template under primer extension conditions but is not perfectly complementary to the template. Numerous factors are known to influence the efficiency and selectivity of hybridization of an oligonucleotide molecule to a second nucleic acid molecule. These factors, which include oligonucleotide length, nucleotide sequence and/or composition, hybridization temperature, buffer composition and potential for steric hindrance in the binding region, should be considered when designing oligonucleotide primers for use in the methods disclosed herein.

Allele-specific primers can be designed for detecting any known or suspected SNP using methods of the present invention. For example, design of allele-specific primers can make use of the approximately 10 million known SNPs. Several databases of SNPs have been established and have steadily been growing in content, including the Human Genome Variation database (HGVbase) (A. J. Brookes et al., Nucl. Acids Res., 2000, 28: 356-360; D. Fredman et al., Nucl. Acids Res., 2002, 30: 387-391), the SNP Consortium (G. A. Thorisson and L. D. Stein, Nucl. Acids Res., 2003, 31: 124-127), and the central database for SNPs (dbSNP) (E. M. Smigielski et al., Nucl. Acids Res., 2000, 28: 352-355). The first high-density map of SNPs comprising features of the human genome was recently created through the combined efforts of the SNP Consortium and the Human Genome Project (R. Sachidanandam et al., Nature, 2001, 409: 928-933). Amino acid sequences and nucleotide sequences of known or suspected SNP sites are available in sequence databases, such as GenBank or the HUGO Mutation Database Initiative, which is establishing a mutational database as a source of common variants of human disease (R. G. Collon et al., Nature, 1998, 279: 10-11). Computer programs, such as Entrez, can be used to browse the databases and retrieve any sequence of interest (see, for example, http://www.ncbi.nlm.nih.gov./Entrez). These databases can also be searched to identify sequences with various degrees of similarity to a query sequence using programs, such as FASTA (W. R. Pearson, Methods Mol. Biol., 2000, 132: 185-219) and BLAST (S. McGinnis and T. L. Madden, Nucl. Acids Res., 2004, 32: W20-25), which rank similar sequences with alignment scores and statistics.

Methods of using allele-specific primers, such as those disclosed herein, have been described (see, for example, C. R. Newton et al., Nucl. Acids Res., 1989, 17: 2503-2516; W. C. Nichols et al., Genomics, 1989, 5: 535-540; D. Y. Wu, Proc. Natl. Acad. Sci. USA, 1989, 86: 2757-2760; C Dutton and S. S. Sommer, Biotechniques, 1991, 11: 700-702; R. S. Cha et al., PCR Methods Appl., 1992, 2: 14-20; L. Ugozzoli and R. B. Wallace, Methods Enzymol., 1991, 2: 42-48).

As will be recognized by one skilled in the art, in the methods of the present invention, a single primer or a set of primers (e.g., forward and reverse primers) can be used depending on whether primer extension, linear amplification or exponential amplification of the template is desired. When a single primer is used, the primer is typically an allele-specific primer, as described herein. When two primers are used, one is an allele-specific primer and the other is a complementary strand primer which anneals to the other DNA strand distant from the allele-specific primer. A set of primer pairs, wherein each pair comprises an allele-specific primer and a complementary strand primer, can also be used to distinguish alleles of a particular SNP. For example, the allele-specific primers of a set can be unique with respect to each other: one of the allele-specific primers may be complementary to the wild-type allele (i.e., allele-specific to the normal allele), and the others may be complementary to the alternative alleles. Each of the allele-specific primers in such a set may be paired with a common complementary strand primer. Multiple sets of pairs of primers can be used for the multiplex detection of SNPs.

### Non-Extendable Oligonucleotide Probes

Certain methods of the present invention include the use of non-extendable oligonucleotide probes in addition to allele-specific extension primers. Non-extendable oligonucleotide probes that can be used in the methods disclosed herein include those described in U.S. Pat. No. 5,849,497 (which is incorporated herein by reference in its entirety).

A suitable non-extendable oligonucleotide probe comprises a target nucleic acid binding domain that forms a stable hybrid with the template DNA at a location 5′ to the termination site. Generally, the target nucleic acid binding domain of a non-extendable oligonucleotide probe comprises at least about 8 and less than about 50 nucleotides. According to certain embodiments of the invention, the target nucleic acid binding domain of a non-extendable oligonucleotide probe comprises at least about 10, at least about 12, at least about 15 or at least about 20 nucleotides. According to certain embodiments, the target nucleic acid binding domain comprises less than about 50, less than about 40, less than about 35 or less than about 30 nucleotides.

Non-extendable oligonucleotide probes for use in methods of the present invention are made non-extendable by adding at least one, and preferably more than one, non-complementary bases at the 3′-end of the target nucleic acid binding domain. For example, at least 2, at least 3, at least 4, at least 5 or more than 5 non-complementary nucleotides may be added at the 3′-tenninus of the target nucleic acid binding domain to make the oligonucleotide probe non-extendable. Under appropriate conditions (such as, for example, in the presence of a DNA polymerase which lacks a 5′→3′ exonuclease activity or both a 5′→3′ and 3′→5′ exonuclease activity), a non-extendable oligonucleotide probe hybridized to the template stops the polymerase, which results in the formation of an extension product of known length. The distance between the 5′ end of the allele-specific primer and the 3′ end of the non-extendable oligonucleotide probe determines the length of the extension product. Thus, if a different extendable oligonucleotide probe is designed for each allele of a single nucleotide polymorphism, the length of the extension product is indicative of which allele is present in the sample. The use of a plurality of non-extendable oligonucleotide probes can allow for multiplex SNP detection.

### Oligonucleotide Preparation

Oligonucleotide primers and probes of the invention may be prepared using any of a variety of methods well-known in the art (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, F. M. Ausubel (Ed.), 5^{th} Ed., 2002, John Wiley & Sons: Secaucus, N.J.). For example, oligonucleotides can be prepared using chemical techniques such as chemical synthesis and polymerization based on a template (S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066).

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of oligonucleotides of the invention, where necessary or desired, may be carried out using any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

If desired, the sequence of synthetic oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), a combination of alkaline phosphatase and exonuclease digestions with mass spectrometry (H. Wu and H. Aboleneen, Anal. Biochem., 2001, 290: 347-352), and the like.

As already mentioned above, in certain embodiments, modified oligonucleotides maybe used in compositions and methods of the present invention. Modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoramidates, carbamates, etc), or charged linkages (e.g., phosphorothioates, phosphorodithioates, etc). Oligonucleotides may contain one or more additional covalently linked moieties, such as, for example, proteins (e.g., nueleases, toxins, antibodies, signal peptides, poly-L-lysine, etc), intercalators (e.g., acridine, psoralen, etc), chelators (e.g., chelators of metals, radioactive metals, oxidative metals, etc) and alkylators. Oligonucleotides may also be derivatized by formation of a methyl or ethyl phosphotriester or an alkyl phosphoramidate linkage. Alternatively or additionally, oligonucleotide sequences of the present invention may be modified with a label.

### Oligonucleotide Labeling

In certain embodiments, the oligonucleotide primers/probes of the present invention are labeled with a detectable agent or moiety before being used in SNP detection assays. The role of a detectable agent is to allow visualization and detection of primer extension products of interest. A label may be directly detectable (i.e., it does not require further reaction or manipulation to be detectable, e.g., a fluorophore is directly detectable) or it may be indirectly detectable (i.e., it is made detectable through reaction or binding with another entity that is detectable; e.g., a hapten becomes detectable after reaction with an appropriate antibody attached to a reporter).

Preferably, the detectable agent is selected such that it generates a signal which can be measured and whose intensity is related (e.g., proportional) to the amount of extension products of interest in the sample being analyzed. In array-based SNP detection methods, the detectable agent is also preferably selected such that it generates a localized signal, thereby allowing spatial resolution of the signal from each spot on the array.

The association between an oligonucleotide primer/probe and a detectable agent can be covalent or non-covalent. Labeled oligonucleotides can be prepared by incorporation of, or conjugation to, a detectable moiety. Labels can be attached directly to the oligonucleotide or indirectly through a linker. Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of monoreactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as, for example, ³²P, ³⁵S, 3H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, i.e., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens, and proteins for which antisera or monoclonal antibodies are available.

In certain embodiments, the oligonucleotide primers of the invention are fluorescently labeled. Numerous known fluorescent labeling moieties of a wide variety of chemical structures and physical characteristics are suitable for use in the practice of this invention. Suitable fluorescent dyes include, but are not limited to fluorescein and fluorescein dyes (e.g., fluorescein isothiocyanine or FITC, naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkyl-aminocoumarin, hydroxycoumarin and aminomethyl-coumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g. Cy-3™, Cy35™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9^{th} Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Berverly, Mass.).

Rather then being directly detectable themselves, some fluorescent groups (donors) transfer energy to another fluorescent group (acceptor) in a process called non-radiative fluorescent resonance energy transfer (FRET), and the second group produces the detectable fluorescent signal. In these embodiments, an oligonucleotide detection probe may, for example, become detectable when hybridized to a primer extension product of interest. Examples of FRET acceptor/donor pairs suitable for use in the present invention include, but are not limited to, fluorescein/tetramethylrhodamine, IAEDANS/FITC, IAEDANS/5-(iodoacetomido)-fluorescein, EDANS/Dabcyl, and B-phycoerythrin/Cy-5.

Detectable moieties can also be biomolecules such as molecular beacons and aptamer beacons. Molecular beacons are nucleic acid molecules carrying a fluorophore and a non-fluorescent quencher on their 5′ and 3′ ends, respectively. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (D. L. Sokol et al., Proc. Natl. Acad. Sci. USA, 1998, 95: 11538-11543; and U.S. Pat. Nos. 6,277,581 and 6,235,504). Aptamer beacons are similar to molecular beacons except that they can adopt two or more conformations (O. K. Kaboev et al., Nucleic Acids Res. 2000, 28: E94; R. Yamamoto et al., Genes Cells, 2000, 5: 389-396; N. Hamaguchi et al., Anal. Biochem. 2001, 294: 126-131; S. K. Poddar and C. T. Le, Mol. Cell. Probes, 2001, 15: 161-167).

A “tail” of normal or modified nucleotides can also be added to the 5′ end of allele-specific oligonucleotide primers for detectability purposes. A second hybridization with a nucleic acid complementary to the tail and containing a detectable label (such as, for example, a fluorophore, an enzyme or bases that have been radioactively labeled) allows visualization of the primer extension products. Alternatively, the nucleic acid complementary to the tail may be attached to a solid surface (e.g., a bead or an array). In certain embodiments of the present invention, the allele-specific oligonucleotide primers are modified to include a tail of normal or modified nucleotides at their 5′ end. The tail may be different for the matched and mismatched allele-specific primers thereby allowing distinction between wild-type and variant SNP.

Selection of a particular nucleic acid labeling technique will depend on the SNP assay to be performed and will be governed by several factors, such as ease and cost of the labeling method, quality of labeling desired, effects of the label on the hybridization reaction (e.g., on the rate and/or efficiency of the hybridization process), nature of the detection system, nature and intensity of the signal generated by the detectable label, and the like.

### II—SNP Detection Methods

As already mentioned above, the present invention provides SNP detection methods which do not require prior amplification of the DNA target sequence(s) that comprise the SNP(s) of interest. In particular, the inventive methods can be used to detect SNPs directly from genomic DNA.

### Sample Preparation

A sample of genomic DNA for use in methods of the present invention may be isolated, extracted or derived from humans, animals, plants, fungi, yeast, bacteria, viruses, tissue cultures, viral cultures, or a combination of the above. In certain embodiments, the sample of genomic DNA to be analyzed according to the invention is isolated, extracted or derived from humans or animals (e.g., mouse, rat, rabbit, dog, cat, cattle, swine, sheep, horse or primate). A sample of genomic DNA may be isolated, extracted or derived from tissues (e.g., bone marrow, lymph nodes, brain, muscles, skin, and the like), body fluids (e.g., serum, blood, urine, sputum, saliva, cerebrospinal fluid, seminal fluid, lymph fluid, and the like), skeletal tissues, or individual cells. A sample of genomic DNA can be isolated, extracted or derived from fetal or embryonic cells or tissues obtained by appropriate methods, such as amniocentesis or chrorionic villus sampling.

Isolation, extraction or derivation of genomic DNA may be carried out by any suitable method. Isolating DNA from a biological sample generally includes treating a biological sample in such a manner that genomic DNA present in the sample is extracted and made available for analysis. Any isolation method that results in extracted/isolated genomic DNA may be used in the practice of the present invention.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual”, 1989, 2^{nd} Ed., Cold Spring Harbour Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300). Several protocols have been developed to extract genomic DNA from blood.

There are also numerous different versatile kits that can be used to extract DNA from tissues and bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

### Fragmentation of Genomic DNA

In certain embodiments, the sample of genomic (unamplified) DNA is submitted to fragmentation before SNP detection. Genomic DNA may be fragmented using any of a variety of methods. Methods of DNA fragmentation are known in the art and include, but are not limited to, DNase digestion, sonication, mechanical shearing, and the like (J. Sambrook et al., “Molecular Cloning: A Laboratory Manual”, 1989, 2^{nd} Ed., Cold Spring Harbour Laboratory Press: New York, N.Y.; P. Tijssen, “Hybridization with Nucleic Acid Probes—Laboratory Techniques in Biochemistry and Molecular Biology (Parts I and II)”, 1993, Elsevier; C. P. Ordahl et al., Nucleic Acids Res., 1976, 3: 2985-2999; P. J. Oefner et al., Nucleic Acids Res., 1996, 24: 3879-3889; Y. R. Thorstenson et al., Genome Res., 1998, 8: 848-855).

In certain embodiments, the sample of genomic DNA is fragmented using ultrasound. The use of sonication to fragment DNA is well-known in the art (H. I. Elsner and E. B. Lindblad, DNA, 1989, 8: 697-701; A. T. Bankier, Methods Mol. Biol., 1993, 23: 47-50; T. L. Mann and U. J. Krull, Biosens. Bioelectron., 2004, 20: 945-955; P. L. Deininger, Anal. Biochem., 1983, 129: 216-223). A generally accepted view is that ultrasound produces a gaseous cavitation (i.e., formation of small bubbles from dissolved gases or vapors due to alteration of pressure in the liquid sample). Fragmentation of DNA is thought to take place, at least in part, as a consequence of mechanical stress or shear from the bubbles leading to breakage of hydrogen bonds and single-strand and double-strand ruptures of the DNA.

In certain methods of the present invention, sonication is carried out under such conditions that the DNA fragments obtained can be used for SNP detection as described herein. Thus, in certain embodiments, sonication is carried out to yield DNA fragments of less than about 2 kilobases (kb) in size, less than about 1.5 kb in size, or less than about 1 kb in size. The energy level, sonication time, temperature and other conditions of sonication to obtain DNA fragments of desired length can readily be determined by a person skilled in the art. Sonication may be performed using any suitable means and instrument including, but not limited to, probe-type sonicators. Probe-type sonicators are commercially available, for example, from Misonix, Inc. (Farmingdale, N.Y.), Sonics & Materials, Inc. (Newtown, Conn.), and Branson Ultrasonics Corp. (Danbury, Conn.).

If desired, the size of the DNA fragments obtained by sonication may be evaluated by any of a variety of techniques such as, for example, gel electrophoresis (B. A. Siles and G. B. Collier, J. Chromatogr. A, 1997, 771: 319-329), sedimentation in gradients, gel exclusion chromatography, or matrix-assisted desorption/ionization time-of-flight (MALDI-TOF) mass spectrometry (N. H. Chiu et al., Nucleic Acids Res., 2000, 28: E31).

### Primer Extension Reaction Mixture

Following optional fragmentation of the DNA test sample, the next step in SNP detection methods of the present invention is to prepare a primer extension reaction mixture, i.e., a composition of matter that includes the elements necessary for a primer extension reaction to occur. In certain embodiments, the template-dependent primer extension reaction is a “high fidelity” reaction. By “high fidelity” reaction it is meant that the reaction has a low error rate, i.e., a low rate of wrong nucleotide incorporation. In these embodiments, the error rate of primer extension reaction is typically less than about 2×10⁻⁴, usually less than about 1×10⁻⁵, and more usually less than about 1×10⁻⁶. In other embodiments, the primer extension reaction is not a high fidelity reaction.

In addition to genomic DNA (e.g., DNA fragments obtained as described above), allele-specific primers and, optionally, non-extendable oligonucleotide probes, the primer extension reaction mixture generally also comprises several other components including deoxyribonucleoside triphosphates (dNTPs), a thermostable nucleic acid polymerase, and an aqueous buffer medium.

Generally, the primer extension reaction mixture will comprise four different types of dNTPs corresponding to the four naturally occurring bases, i.e., dATP, dTTP, dCTP, and dGTP. In certain embodiments, the primer extension mixture additionally contains biotinylated dNTPs, for example biotinylated dCTP, for incorporation of biotin in the primer extension product. The resulting biotinylated primer extension products may subsequently be exposed to a streptavidin-dye complex for detection purposes, as is well-known in the art. Examples of streptavidin-dye complexes suitable for use in the practice of methods of the present invention include, but are not limited to, steptavidin-fluorescein (SA-FITC), streptavidin-phycoerythrin (SA-PE), streptavidin-rhodamine B (SA-R), streptavidin-Texas Red (SA-TR), streptavidin-phycocyanin (SA-PC), and streptavidin-allophycocyanine (SA-APC).

The primer extension reaction mixture generally also comprises a thermostable nucleic acid polymerase. As used herein, the term “thermostable” refers to an enzyme which is stable and active at a temperature as great as between about 90° C. and about 100° C., or between about 70° C. and about 98° C. A representative thermostable nucleic acid polymerase isolated from Thermus aquaticus (Taq) is described in U.S. Pat. No. 4,889,818 and a method for using it in conventional PCR is described in R. K. Saiki et al., Science, 1988, 239: 487-491. Another representative thermostable nucleic acid polymerase isolated from P. furiosus (Pfu) is described in K. S. Lundberg et al., Gene, 1991, 108: 1-6. Additional examples of thermostable polymerases include polymerases extracted from the thermophilic bacteria Thermus flavus, Thermus ruber, Thermus thermophilus, Bacillus stearothermophilus, Thermus lacteus, Thermus rubens, Thermotoga maritima, or from thermophilic archaea Thermococcus litoralis and Methanothermus fervidus.

Thermostable DNA polymerases suitable for use in the practice of the present invention include, but are not limited to, E. coli DNA polymerase I, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

In certain embodiments, the primer extension reaction mixture comprises a thermostable nucleic acid polymerase lacking 5′→3′ exonuclease activity or lacking both 5′→3′ and 3′→5′ exonuclease activity. More specifically, an important aspect of the methods of the present invention includes the use of an exonuclease-deficient polymerase for extension of the primer strand formed by the allele-specific oligonucleotide and using the target DNA as a template for extending this allele-specific primer in a manner such that no extension occurs if there is a mismatch at the terminal 3′ end of the allele-specific primer.

Examples of nucleic acid polymerases substantially lacking 5′→3′ exonuclease activity include, but are not limited to, Klenow and Klenow exo-, and T7 DNA polymerase (Sequenase). Examples of thermostable nucleic acid polymerases substantially lacking 5′→3′ exonuclease activity include, but are not limited to, Pfu, the Stoffel fragment of Taq, N-truncated Bst, N-truncated Bca, Genta, JdF3 exo, Vent, Deep Vent, U1Tma and ThermoSequenase. Examples of thermostable nucleic acid polymerases substantially lacking both 5′→3′ and 3′→5′ exonuclease activity include, but are not limited to, exo-Pfu (a mutant form of Pfu), Vent exo (a mutant form of Vent), and Deep Vent exo- (a mutant form of Deep Vent).

Thermostable nucleic acid polymerases are commercially available for example from Stratagene (La Jolla, Calif.), New England BioLabs (Ipswich, Mass.), BioRad (Hercules, Calif.), Perkin-Elmer (Wellesley, Mass.), and Hoffman-LaRoche (Basel, Switzerland).

The primer extension reaction mixture generally comprises enough thermostable polymerase such that conditions suitable for enzymatic primer extension are maintained during all the reaction cycles. Alternatively, polymerase may be added to the primer extension reaction mixture after a certain number of reaction cycles have been performed.

The primer extension reaction mixture generally further comprises an aqueous buffer medium which acts as a source of monovalent ions, divalent cations, and a buffer agent. Any convenient source of monovalent ions, such as potassium chloride, potassium acetate, potassium glutamate, ammonium acetate, ammonium chloride, ammonium sulfate, and the like may be employed. The divalent cation may be magnesium, manganese, zinc and the like. Magnesium (Mg²⁺) is often used. Any source of magnesium cations may be employed, including magnesium chloride, magnesium acetate, and the like. The amount of Mg²⁺ present in the buffer may range from about 0.5 to about 10 mM. Representative buffering agents, or salts that may be present in the buffer include Tris, Tricine, HEPES, MOPS, and the like. The amount of buffering agent generally ranges from about 5 mM to about 150 mM. In certain embodiments, the buffer agent is present in an amount sufficient to provide a pH ranging from about 6.0 to about 9.5, most preferably about pH 7.3. Other agents which may be present in the buffer medium include chelating agents, such as EDTA, EGTA and the like.

In preparing the primer extension reaction mixture, the various constituent components may be combined in any convenient order.

### Primer Extension Reaction

Following addition/combination of all the components, the reaction mixture is subjected to primer extension reaction conditions, i.e., to conditions that allow for polymerase-mediated primer extension by addition of nucleotides to the end of the annealed (i.e., hybridized) primer molecule using the target strand as a template.

In many embodiments, the primer extension reaction conditions are PCR amplification conditions. The PCR (or polymerase chain reaction) technique is well-known in the art and has been disclosed in K. B. Mullis and F. A. Faloona, Methods Enzymol., 1987, 155: 355-350 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest (i.e., the region to be amplified) in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). In the absence of a non-extendable oligonucleotide probe, the termini of the amplified fragments are defined by the 5′ ends of the primers. In the presence of a non-extendable oligonucleotide probe, the termini of the amplified fragments are defined by the 5′ end of the allele-specific primer and the 3′ end of the oligonucleotide probe.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art.

Although the number of reaction cycles may vary depending on the detection analysis being performed, it usually is at least about 15, more usually at least about 20, and may be as high as about 60 or higher. However, in many situations, the number of reaction cycles typically range from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about 75° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions suitable for polymerization of nucleotides to the primer's end in a manner such that the primer is extended in a 5′ to 3′ direction using the DNA to which it is hybridized as a template, (i.e., conditions suitable for enzymatic formation of a primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described, for example, in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Other methods of enzymatic nucleic acid amplification that can be used in primer extension reactions include, but are not limited to, Transcription-Mediated Amplification (or TMA, described in, for example, D. Y. Kwoh et al., Proc. Natl. Acad. Sci. USA, 1989, 86: 1173-1177; C. Giachetti et al., J. Clin. Microbiol., 2002, 40: 2408-2419; and U.S. Pat. No. 5,399,491); Self-Sustained Sequence Replication (or 3SR, described in, for example, J. C. Guatelli et al., Proc. Natl. Acad. Sci. USA, 1990, 87: 1874-1848; and E. Fahy et al., PCR Methods and Applications, 1991, 1: 25-33); Nucleic Acid Sequence Based Amplification (or NASBA, described in, for example, T. Kievits et al., J. Virol., Methods, 1991, 35: 273-286; and U.S. Pat. No. 5,130,238) and Strand Displacement Amplification (or SDA, described in, for example, G. T. Walker et al., PNAS, 1992, 89: 392-396; EP 0 500 224 A2). Each of the references cited in this paragraph is incorporated herein by reference in its entirety.

### Analysis of Primer Extension Products

Analysis of primer extension products can be accomplished by any of a wide variety of methods.

Following primer extension, it may be desirable to separate the primer extension products from each other and from other components of the extension reaction mixture (e.g., DNA fragments including template, excess primers/probes, etc) for purpose of analysis. As already mentioned above, in certain multiplex SNP detection methods of the present invention, a plurality of non-extendable oligonucleotide probes are used, wherein each non-extendable oligonucleotide probe is designed for a particular target and generates primer extension products of a particular size, ultimately resulting in the formation of extended fragments of different sizes, each size being characteristic of a particular SNP.

Thus, in certain embodiments, separation of primer extension products from other components of the extension reaction mixture is accomplished using methods that achieve separation of DNA fragments on the basis of length, size, mass, charge or any other physical property of the primer extension products. Such methods are well-known in the art and include, but are not limited to, chromatographic methods (including, for example, liquid chromatography such as high performance liquid chromatography or HPLC), electrophoretic methods (such as gel electrophoresis and capillary electrophoresis), and mass spectrometry methods (including, for example, electrospray/ionspray (ES) and matrix-assisted laser desorption/ionization (MALDI-TOF) spectrometry techniques).

In some embodiments, separation of primer extension products from other components of the reaction mixture is accomplished by employing capture reagents. Capture reagents typically consist of a solid support material coated with one or more binding members specific for the same or different binding partners. The term “solid support material”, as used herein, refers to any material which is insoluble or can be made insoluble by a subsequent reaction or manipulation. Solid support materials can be latex, plastic, derivatized plastic, magnetic or non-magnetic metal, glass or silicon surface or surfaces of test tubes, microtiter wells, sheets, beads, microparticles, chips and other configurations known to those of ordinary skill in the art. To facilitate separation and/or detection of primer extension products, an extension primer can be labeled with a binding member that is specific for its binding partner, which binding partner is attached to a solid material. The primer extension products can be separated from other components of the extension reaction mixture by contacting the mixture with a solid support, and then removing, from the reaction mixture, the solid support to which extension products are bound, for example, by filtration, sedimentation, washing or magnetic attraction.

For example, an allele-specific extension primer can be coupled with a moiety that allows affinity capture, while other allele-specific primers remain unmodified or are coupled with different affinity moieties. Modifications can include a sugar (for binding to a solid phase material coated with lectin), a hydrophobic group (for binding to a reverse phase column), biotin (for binding to a solid phase material coated with streptavidin), or an antigen (for binding to a solid phase material coated with an appropriate antibody). Extension reaction mixtures can be run through an affinity column, the flow-through fraction collected, and the bound fraction eluted, for example, by chemical cleavage, salt elution, and the like. Alternatively, extension reaction mixtures can be contacted with affinity capture beads.

Alternatively, each extension primer may comprise a nucleotide sequence (binding member) at its 5′ terminus, that is complementary to a nucleotide sequence (binding partner) attached to a solid support. The extension primers used in a SNP detection method of the present invention may be coupled to an identical tag sequence (e.g., universal capture sequence) complementary to a tag probe sequence attached to a solid support. Alternatively, each extension primer used in an inventive SNP detection method may comprise a tag sequence that is allele-specific and complementary to a tag probe sequence attached to a solid support. The tag may be, for example, about 10 to about 30 nucleotides in length. Tags and specific sets of tag and tag probe sequences are disclosed for example, in U.S. Pat. No. 6,458,530 (which is incorporated herein by reference in its entirety). In general, tag and tag probe sequences are selected such that they are not present in the genome (or part of the genome) of interest in order to prevent cross-hybridization with the genome. Tags are often selected in sets; and tags in a set are generally selected such that they do not cross-hybridize with another tag or complement of another tag within the set. Tag probe sequences may be attached to multiple microspheres/microparticles or to an array or micro-array. An array or micro-array may be prepared to contain a plurality of probe elements. For example, each probe elements may include a plurality of tag probes that comprise substantially the same sequence that may be of different lengths. Probe elements on an array may be arranged on the solid surface at different densities.

Methods of attaching (or immobilizing) tag sequences to a solid support are known in the art (see, for example, J. Sambrook et al., “Molecular Cloning: A Laboratoty Manual”, 1989, 2^{nd} Ed., Cold Spring Harbour Laboratory Press: New York, N.Y.; “Short Protocols in Molecular Biology”, 2002, F. M. Ausubel (Ed.), 5^{th} Ed., John Wiley & Sons; U. Maskos and E. M. Southern, Nucleic Acids Res. 1992, 20: 1679-1684; R. S. Matson et al., Anal. Biochem. 1995, 224; 110-116; R. J. Lipshutz et al., Nat. Genet. 1999, 21: 20-24; Y. H. Rogers et al., Anal. Biochem. 1999, 266: 23-30; M. A. Podyminogin et al., Nucleic Acids Res. 2001, 29: 5090-5098; Y. Belosludtsev et al., Anal. Biochem. 2001, 292: 250-256; U.S. Pat. Nos. 5,427,779, 5,512,439, 5,589,586, 5,716,854 and 6,087,102). Alternatively, one can rely on commercially available systems including arrays and microarrays, such as those developed, for example, by Affymetrix, Inc. (Santa Clara, Calif.) and Illumina, Inc. (San Diego, Calif.); and multiplexed bead- and particle-based systems such as those developed by BD Biosciences (Bedford, Mass.) and Luminex, Corp. (Austin, Tex.).

After separation from other components of the extension reaction mixture, the presence or absence of extension products (indicative of the presence or absence of particular SNPs in the genomic DNA sample under investigation) can be detected using any of a wide variety of methods, including spectroscopic, photochemical, biochemical, immunochemical, electrical, optical, radiochemical, and chemical methods. Selection of a method of detection will generally depend on several factors including, but not limited to, the type of assay carried out (e.g., single-plex vs. multi-plex; homogeneous vs. heterogeneous), the presence or absence of a label (i.e., detectable moiety) on the extension products, and the nature of these labels (e.g., directly vs. indirectly detectable), if present.

For example, if primer extension products are separated using a mass spectrometry technique, the extension products are detected directly and identified through their mass. Primer extension products separated using an electrophoretic method (e.g., capillary electrophoresis) or a chromatographic method (e.g., HPLC) may be detected by absorption of UV light, a property inherent to DNA, or by fluorescence if the primer extension products are fluorescently labeled. In these methods, the detection is dynamic (i.e., each extension product is detected as it moves past a detector). As known in the art, extension products separated by polyacrylamide gel or slab gel electrophoresis can easily be detected if they contain a fluorophore, a chromophore or a radioisotope. Primer extension products separated by polyacrylamide gel or slab gel electrophoresis can, alternatively, be detected by associated enzymatic reaction. Enzymatic reaction involves binding an enzyme to a product (e.g., via biotin/avidin interaction) following separation of the primer extension products on a gel, and then detecting the enzyme-labeled product by chemical reaction, such as chemiluminescence generated with luminol.

Primer extension products generated by methods of the present invention may be indirectly detected through hybridization. For example, the extension products may be contacted with labeled nucleic acid probes. Hybridization of an extension product to a labeled nucleic acid probe allows visualization of the extension product. For example, each nucleic acid probe may be specific for an extension product (indicative of one allele of a SNP of interest) and may be labeled with a detectable moiety that is different from the detectable moieties carried by the other nucleic acid probes used in the assay, thereby allowing multiplex SNP detection.

Nucleic acid probes may be conjugated to a fluorescent dye, a chromophore, a radioisotope, a mass label (see, for example U.S. Pat. Nos. 5,003,059; 5,547,835; 6,312,893; and 6,623,928), or a binding member such as an antibody or biotin, where the other member of the binding pair (for example, antigen or avidin, respectively) carries a detectable moiety. Alternatively, nucleic acid probes may be labeled with acridinium ester (AE), a highly chemiluminescent molecule (Weeks et al., Clin. Chem., 1983, 29: 1474-1479; Berry et al., Clin. Chem., 1988, 34: 2087-2090) using a non-nucleotide-based linker arm chemistry (U.S. Pat. Nos. 5,585,481 and 5,185,439). Detection includes triggering chemiluminescence by AE hydrolysis with alkaline hydrogen peroxide, which yields an excited N-methyl acridone that subsequently deactivates with emission of a photon. In the absence of a target sequence (i.e., extension product), AE hydrolysis is rapid. However, the rate of AE hydrolysis is greatly reduced when the probe is bound to the extension product. Thus, hybridized and un-hybridized AE-labeled probes can be detected directly in solution, without the need for physical separation. Alternatively, the labeled nucleic acid probes may be TaqMan™ (U.S. Pat. Nos. 5,210,015; 5,804,375; 5487,792 and 6214,979) or Molecular Beacon™ (S. Tyagi and F. R. Kramer, Nature Biotechnol. 1996, 14: 303-308; S. Tyagi et al., Nature Biotechnol. 1998, 16: 49-53; L. G. Kostrikis et al., Science, 1998, 279: 1228-1229; D. L. Sokol et al., Proc. Natl. Acad. Sci. USA, 1998, 95: 11538-11543; S. A. Marras et al., Genet. Anal. 1999, 14: 151-156; and U.S. Pat. Nos. 5,846,726, 5,925,517, 6,277,581 and 6,235,504) probes. Using the latter detection methods, extension products can be detected as they are formed or in a so-called real time manner.

Extension products bound to microspheres (also called microparticles or microbeads) can be detected using different methods. For example, in multiplexed assays of the present invention, extension products can be simultaneously detected using pre-coded microbeads. Beads may be pre-coded using specific bead sizes, different colors and/or color intensities, different fluorescent dyes or fluorescent dye combinations.

Color-coded microspheres can be made using any of a variety of methods such as those disclosed in U.S. Pat. Nos. 6,649,414; 6,514,295; 5,073,498; 5,194,300; 5,356,713; 4,259,313; 4,283,382 and the references cited in these patents. Color-coded microspheres are also commercially available, for example, from Cortex Biochem., Inc. (San Leandro, Calif.); Seradyn, Inc. (Indianapolis, Ind.); Dynal Biotech, LLC (Brown Deer, Wis.); Spherotech, Inc. (Libertyville, Ill.); Bangs Laboratories, Inc. (Fishers, Ind.); and Polysciences, Inc. (Warrington, Pa.).

For example, polystyrene microspheres are provided by Luminex Corp. (Austin, Tex.) that are internally dyed with two spectrally distinct fluorescent dyes (x-MAP™ microbeads). Using precise ratios of these fluorophores, a large number of different fluorescent bead sets can be produced (e.g., 100 sets). Each set of beads can be distinguished by its code (or spectral signature), a combination of which allows for detection of a large number of different extension products in a single reaction vessel. The magnitude of the biomolecular interaction that occurs at the microsphere surface is measured using a third fluorochrome that acts as a reporter. These sets of fluorescent beads with distinguishable codes can be used to label extension products. Labeling (or attachment) of extension products to beads can be by any suitable means including, but not limited to, chemical or affinity capture, cross-linking, electrostatic attachment, and the like. In certain embodiments, labeling is carried out through hybridization of allele-specific tag and tag probe sequences, as described above. Because each of the different extension products is uniquely labeled with a fluorescent bead, the captured extension product (indicative of one allele of a SNP of interest) will be distinguishable from other different extension products (including extension products indicative of other alleles of the same SNP and extension products indicative of other SNPs of interest). Following tag/tag probe hybridization, the microbeads can be analyzed using different methods such as, for example, flow cytometry-based methods.

Flow cytometry is a sensitive and quantitative technique that analyzes particles in a fluid medium based on the particles' optical characteristics (H. M. Shapiro, “Practical Flow Cytometry”, 3^{rd} Ed., 1995, Alan R. Liss, Inc.; and “Flow Cytometry and Sorting, Second Edition”, Melamed et al. (Eds), 1990, Wiley-Liss: New York). A flow cytometer hydrodynamically focuses a fluid suspension of particles containing one or more fluorophores, into a thin stream so that the particles flow down the stream in a substantially single file and pass through an examination or analysis zone. A focused light beam, such as a laser beam, illuminates the particles as they flow through the examination zone, and optical detectors measure certain characteristics of the light as it interacts with the particles (e.g., light scatter and particle fluorescence at one or more wavelengths). In the stream, the microbeads are interrogated individually as they pass the detector and high-speed digital signal processing classifies each bead based on its code and quantifies the reaction on the bead surface. A large number of beads can be interrogated per second, resulting in a high-speed, high-throughput and accurate detection of multiple different SNPs. In embodiments where the primer extension reaction is carried out in the presence of biotinylated dNTPs, the reaction between beads and extension products may be quantified by fluorescence after reaction with fluorescently-labeled streptavidin (e.g., Cy5-streptavidin conjugate). Instruments for performing such assay analyses are commercially available, for example, from Luminex (e.g., Luminex® 100™ Total System, Luminex® 100™ IS Total System, Luminex® High Throughput Screening System).

Alternatively or additionally, the microbeads can be distributed in or on an additional support or substrate, such as a micro-well plate or an array.

Extension products bound to arrays, micro-arrays or chips can be detected using different methods. In certain embodiments, primer extension products are captured (or attached) via hybridization to probes on array sites (as mentioned above). This attachment is generally a direct hybridization between an adapter sequence on the primer extension product (e.g., an allele-specific tag sequence) and a corresponding capture probe (e.g., complementary tag probe sequence) immobilized onto the surface of the array. Alternatively, the attachment can rely on indirect “sandwich” complexes using capture extender probes as known in the art (see, for example, M. Ranki et al., Gene, 1983, 21: 77-85; B. J. Connor et al., Proc. Natl. Acad. Sci. USA, 1983, 80: 278-282; and U.S. Pat. Nos. 4,563,419 and 4,751,177). The presence or absence of a bound extension product at a given spot (or position) on the array is generally determined by detecting a signal (e.g., fluorescence) from the label coupled to the product. Furthermore, since the sequence of the capture probe at each position on the array is known, the identity of an extension product at that position can be determined.

Extension products bound to arrays are often (directly or indirectly) fluorescently detected. Methods for the detection of fluorescent labels in array configurations are known in the art and include the use of “array reading” or “scanning” systems, such as charge-coupled devices (i.e., CCDs). Any known device or method, or variation thereof can be used or adapted to practice methods of the invention (see, for example, Y. Hiraoka et al., Science, 1987, 238: 36-41; R. S. Aikens et al., Meth. Cell Biol. 1989, 29: 291-313; A. Divane et al., Prenat. Diagn. 1994, 14: 1061-1069; S. M. Jalal et al., Mayo Clin. Proc. 1998, 73: 132-137; V. G. Cheung et al., Nature Genet. 1999, 21: 15-19; see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarray scanners are typically laser-based scanning systems that can acquire one (or more than one) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during acquisition of the fluorescent images. Filter sets are commercially available, for example, from Chroma Technology Corp. (Rockingham, Vt.).

A computer-assisted image analysis system is generally used to analyze fluorescent images acquired from arrays. Such systems allow for an accurate quantitation of the intensity differences and for an easy interpretation of the results. A software for fluorescence quantitation and fluorescence ratio determination at discrete spots on an array is usually included with the scanner hardware. Softwares and/or hardwares are commercially available and may be obtained from, for example, Affymetrix, Inc. (Santa Clara, Calif.), Applied Spectral Imaging, Inc. (Carlsbad, Calif.), Chroma Technology Corp. (Rockingham, Vt.), Leica Microsystems (Bannockburn, Ill.), and Vysis, Inc. (Downers Grove, Ill.).

Alternatively, a planar waveguide (PWG) chip technique can be used to detect surface-bound fluorescently-labeled extension products. A waveguide refers to a two dimensional total internal reflection (TIR) element that provides an interface capable of internal reference at multiple points, thereby creating an evanescent wave that is substantially uniform across all or nearly all the entire surface. The waveguide can be comprised of transparent material such as glass, quartz, plastics such as polycarbonate, acrylic or polystyrene. The glass or other types of surfaces used for waveguides can be modified with any of a variety of functional groups including binding members such as haptens or oligonucleotide sequences (e.g., tag probe sequences).

In PWG, fluorescent excitation is carried out using an exponentially decaying evanescent light field, which preferentially excites labeled molecules that are captured within the field. Since molecules in solution (i.e., non surface bound) are not within the evanescent field, they do not get excited. This technique presents several advantages including very low fluorescent background, and high dynamic range, and allows measurements in turbid solutions or optically dense suspensions. Multiplexed detection can be achieved by combining 2D arrays of ligands and CCD camera detection.

As will be appreciated by one skilled in the art, extension products generated using methods of the present invention may be detected using any other suitable technique that those described above.

### III. Applications of the Inventive SNP Detection Methods

The methods of the present invention can be used in a wide variety of applications, including, but not limited to, correlation of genotype information to phenotype, disease susceptibility, disease diagnosis, pharmacogenomics (i.e., tailoring of drug therapy to an individual's genotype), design and development of new drugs, human identification such as in forensics, paternity testing, and population genetics studies.

Correlation of SNPs with Phenotypic Traits

SNP genotyping for disease diagnosis, disease predisposition screening, disease prognosis, determination of drug responsiveness, drug toxicity screening, and other uses such as those described herein, typically relies on initially establishing a genetic association between one or more SNPs and the particular phenotypic trait of interest. Phenotypic traits include diseases that have known but hitherto unmapped genetic components (e.g., diabetes insipidus, Lesh-Nyhan syndrome, muscular dystrophy, familial hypercholesterolemia, polycystic kidney disease, von Willebrand's disease, tuberous sclerosis, familial colonic polyposis, osteogenesis imperfecta, and acute intermittent porphyria). Phenotypic traits also include symptoms of, or susceptibility to, multifactorial diseases of which a component is or may be genetic, such as autoimmune diseases, inflammation, cancer, diseases of the nervous system, and infection by pathogenic microorganisms. Examples of autoimmune diseases include rheumatoid arthritis, multiple sclerosis, diabetes, systemic lupus erythematosus and Graves disease. Examples of cancers include cancer of the bladder, brain, breast, colon, esophagus, kidney, liver, lung, oral cavity, ovary, pancreas, prostate, skin, stomach, and uterus. Phenotypic traits also include characteristics such as longevity, appearance (e.g., baldness, obesity), strength, speed, endurance, fertility, and susceptibility to particular drugs or therapeutic treatments.

To identify a correlation between one or more alleles and one or more phenotypic traits, individuals are tested for the presence or absence of polymorphic SNP markers or SNP marker sets and for the phenotypic trait or traits of interest. The presence or absence of a set of SNPs is compared for individuals who exhibit a particular trait (e.g., certain manifestations of a disease) and individuals who lack the particular trait to determine if the presence or absence of a particular allele (or combination of alleles) is associated with the trait of interest.

Generally, in a genetic association study, tissue specimens (e.g., whole blood) from the sampled individuals may be collected and genomic DNA genotyped for the SNPs of interest. In addition to the phenotypic trait of interest, other information such as demographic (e.g., age, gender, ethnicity, etc), clinical and environmental information that may influence the outcome of the trait can be collected to further characterize and define the sample set. After all the relevant phenotypic and genotypic information has been obtained, statistical analyses are carried out to determine if there is any significant correlation between the presence of an allele or a genotype with the phenotype characteristics of an individual.

### Disease Diagnosis, Disease Prognosis, and Disease Predisposition

The correlation or association of particular SNPs with disease phenotypes, such as human disease, can be used to develop diagnostic tests capable of identifying individuals who express a detectable trait, such as human disease, as the result of a specific genotype or individuals whose genotype places them at risk of developing a detectable trait at a subsequent time. The diagnostics may be based on a single SNP or a group of SNPs. As will be recognized by one skilled in the art, combined detection of a plurality of SNPs typically increases the probability of an accurate diagnosis. To further increase the accuracy of diagnosis or susceptibility screening, analysis of SNPs according to methods of the present invention can be combined with analysis of other risk factors of human disease, such as family history, diet or lifestyle factors.

To diagnose disease or pre-disposition to disease, individuals are tested for the presence or absence of one or more SNPs that correlate with one or more diseases. Individuals can be tested before symptoms of the disease develop. Infants, for example, can be tested for genetic diseases before birth or at birth. Individuals of any age can be tested to determine risk profiles for the occurrence of future disease. Often early diagnosis can lead to more effective treatment and prevention of disease through dietary, behavior or pharmaceutical interventions. Individuals can be tested to determine carrier status for genetic disorders. Potential parents can use this information to make family planning decisions.

Individuals who develop symptoms of disease that are consistent with more than one diagnosis can be tested to make a more accurate diagnosis. For example, genetic expression information discovered through the use of arrays has been used to determine the specific type of cancer affecting a particular patient (Golub et al., Science, 2001, 286: 531-537; Yeoh et al., Cancer Cell, 2002, 1: 133-143; Armstrong et al., Nature Genetics, 2002, 30: 41-47)

### Pharmacogenomics

SNP analysis according to the present invention can also be used in pharmacogenomics. Pharmacogenomics examines the inherited genetic variations that dictate drug responses and explores ways in which these variations can be used to predict how a patient will respond to medications (A. D. Roses, Nature, 405: 857-865; M. Eichelbaum and B. Evert, Clin. Exp. Pharmacol. Physiol., 1996, 23: 983-985; M. W. Linger et al., Clin. Chem., 1997, 43: 254-266). Thus, pharmacogenomics can enhance and optimize the therapeutic effectiveness of a treatment by allowing physicians to select effective drugs and effective dosage regimens of these drugs based on a patient's SNP genotype. Furthermore, pharmacogenomics can decrease the likelihood of adverse effects by allowing physicians to identify individuals predisposed to toxicity and adverse reactions to particular drugs and drug dosages.

Pharmacogenomics is also of great interest to pharmaceutical companies, as it provides means to decrease time and cost of drug development and to reduce failure rates. In particular, SNP genotyping methods according to the present invention can be used advantageously to shorten and reduce costs of clinical trials by allowing pre-selection of individuals with particular genotypes. In addition, pharmacogenomics can provide greater incentive to pharmaceutical companies to pursue research into drugs that are highly effective for only a very small percentage of the population, while proving only slightly effective or even ineffective to a large percentage of patients, and/or into drugs which, while being highly effective to a large percentage of the population, prove dangerous or even lethal for a small percentage of the population.

### Paternity Testing and Determination of Relatedness

There are many circumstances where relatedness between individuals is the subject of genotype analysis and methods of the present invention can be applied to these procedures.

Paternity testing is commonly used to establish whether a male is the father of a child. In most cases, the mother of the child is known and thus, the mother's contribution to the child's genotype can be traced. Paternity testing investigates whether the part of the child's genotype not attributable to the mother is consistent with that of the putative father. Genetic material from the child can be analyzed for occurrence of one or more SNPs and compared to a similar analysis of the putative father's genetic material. If the set of SNPs in the child does not match the set of SNPs of the putative father, it can be concluded, barring experimental error, that the putative father is not the real biological father. If the set of SNPs in the child attributable to the father does not match the set of SNPs of the putative father, a statistical calculation can be performed to determine the probability of coincidental match.

Determination of relatedness is not limited to the relationship between father and child, but can also be done to determine the relatedness between mother and child, or more broadly, to determine how related one individual is to another, for example, between races or species, or between individuals from geographically separated populations (H. Kaessmann et al., Nature Genet., 1999, 22: 78).

### Forencics

SNPs are also markers of choice in forensic applications. In particular, compared to other markers (e.g., STR markers), SNPs are much shorter. This makes SNPs more amenable to typing in highly degraded or aged biological samples that are frequently encountered in forensic casework in which DNA may be fragmented into short pieces. DNA can be isolated from biological samples such as blood, bone, hair, saliva, and semen and compared with the DNA of a reference source or a criminal DNA databank at particular SNP positions. Methods of the present invention can be used to assay simultaneously multiple SNP markers in order to decrease the power of discrimination and the statistical significance of a matching genotype.

As will be recognized by those skilled in the art, the inventive methods can also find applications in other fields than those described herein. For example, they can be used as screening tools to accelerate the selective breeding process in agriculture or the selection of desirable trait(s) in model organisms for research, or to characterize biological threat agents in environmental samples.

### IV-Kits

In another aspect, the present invention provides kits comprising materials useful for the detection of one or more SNPs in unamplified genomic DNA according to methods disclosed herein. The inventive kits may be used by diagnostic laboratories, clinical laboratories, experimental laboratories, or practitioners. The invention provide kits which can be used in such settings.

Basic materials and reagents for detection of SNPs according to the present invention may be assembled together in a kit. An inventive kit comprises at least one set of primers (e.g., comprising one matched allele-specific primer and one mismatched allele-specific primer) and, optionally, a non-extendable oligonucleotide probe. Each kit necessarily comprises the reagents which render the procedure specific. Thus, a kit intended to be used for the detection of a particular SNP preferably comprises a matched and mismatched allele-specific primers set specific for the detection of that particular SNP, and optionally, a non-extendable oligonucleotide probe. A kit intended to be used for the multiplex detection of a plurality of SNPs comprises a plurality of primer sets, each set specific for the detection of one particular SNP, and, optionally, a plurality of corresponding non-extendable oligonucleotide probes.

In certain embodiments, the inventive kits further comprise at least one set of pre-selected nucleic acid sequences that act as capture probes for the extension products. The pre-selected nucleic acid sequences may be immobilized on an array or beads (e.g., coded beads).

The inventive kit may further comprise amplification reagents. Suitable amplification reaction reagents include, for example, one or more of: buffers, reagents, enzymes having polymerase activity; enzymes having polymerase activity and lacking 5′→3′ exonuclease activity or both 5′→3′ and 3′→5′ exonuclease activity; enzyme cofactors such as magnesium or manganese; salts; deoxynucleoside triphosphates (dNTPs); biotinylated dNTPs, suitable for carrying out the amplification reaction.

The kit may further comprise one or more of: wash buffers and/or reagents, hybridization buffers and/or reagents, labeling buffers and/or reagents, and detection means. The buffers and/or reagents are preferably optimized for the particular amplification/detection technique for which the kit is intended. Protocols for using these buffers and reagents for performing different steps of the procedure may also be included in the kit.

Kits may also contain reagents for the isolation of genomic DNA from biological samples prior to primer extension.

The reagents may be supplied in a solid (e.g., lyophilized) or liquid form. The kits of the present invention optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kits may also comprise instructions for using the amplification reaction reagents, sets of primers, sets of pre-selected nucleic acid sequences and non-extendable oligonucleotide probes according to the present invention. Instructions for using a kit according to one or more methods of the invention may comprise instructions for processing the biological sample, extracting genomic DNA from the biological sample, fragmenting the genomic DNA by sonication, and/or performing the test; instructions for interpreting the results as well as a notice in the form prescribed by a governmental agency (e.g., FDA) regulating the manufacture, use or sale of pharmaceuticals or biological products.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Some of the results reported in this section have been presented at the American Association for Clinical Chemistry's annual Oak Ridge Conference on Apr. 14 and 15, 2005 (Baltimore, Md.) and described in an abstract (Abstract 71: J. Burmeister et al., “Single Nucleotide Polymorphism Analysis Without Target Amplification on Planar Waveguides”), which is incorporated herein by reference in its entirety.

Cystic Fibrosis disease results from mutations in the cystic fibrosis transmembrane conductance regulator (CFTR) gene, which is located on the long arm of chromosome 7. In the present study, six SNPs (G542X, G551D, 1717-1G>A, R560T, R1162× and 3659delC) from the CFTR gene were chosen as a model system. The closer the mutations are to one another the more difficult they are to detect.

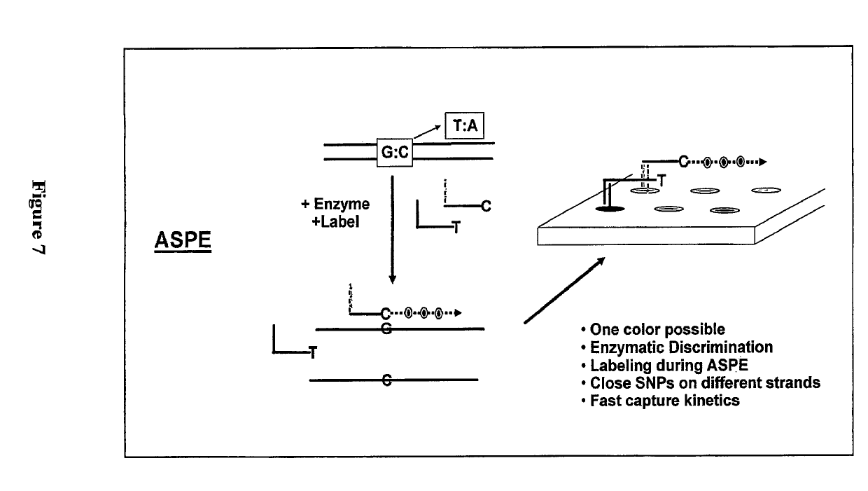

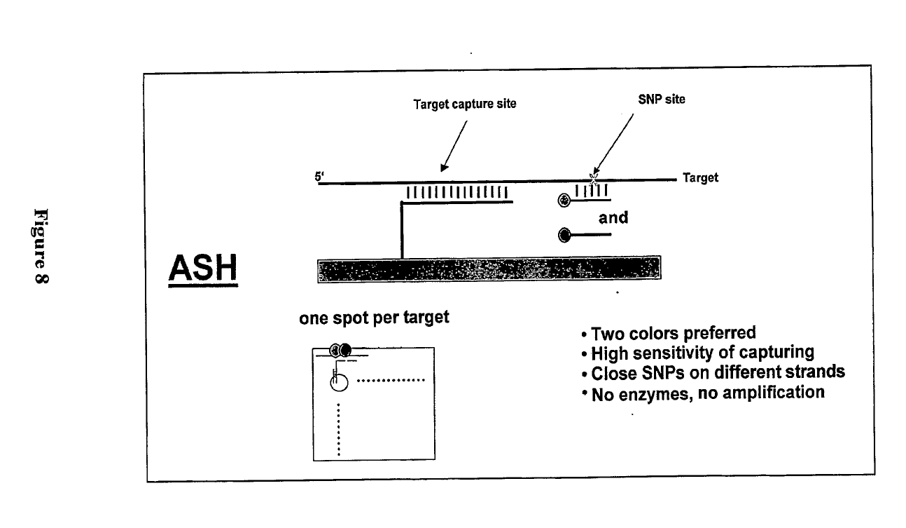

Two different assay configurations were implemented: allele-specific primer extension (ASPE), according to the present invention (see FIG. 7), and allele-specific hybridization (ASH, see FIG. 8). For ASPE, universal capture sequences were arrayed on planar waveguide chips. These universal sequences were complementary to sequence overhangs on the 5′-termini of the respective allele-specific primers.

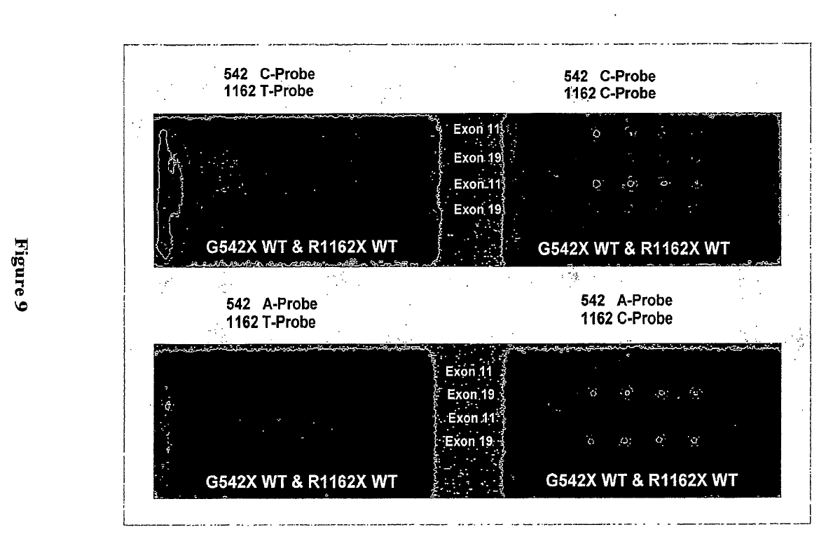

Planar waveguide (PWG) chip technology allows for high sensitivity detection of surface bound, fluorescently labeled analytes. Fluorescent excitation by an exponentially decaying evanescent light field preferentially excites labeled molecules that are captured within the field. Molecules that are in solution are not within the evanescent field and do not get excited. Fluorescent backgrounds are very low, dynamic range is high, and measurements in turbid solutions or optically dense suspensions such as whole blood are possible. Multiplexed detection can be achieved by combining 2D arrays of ligands and inexpensive CCD camera detection. The PGW's high sensitivity was exploited to directly analyze genomic samples for single nucleotide polymorphisms without prior target amplification according to the present invention. FIG. 9 shows data obtained using Planar Waveguide technology.

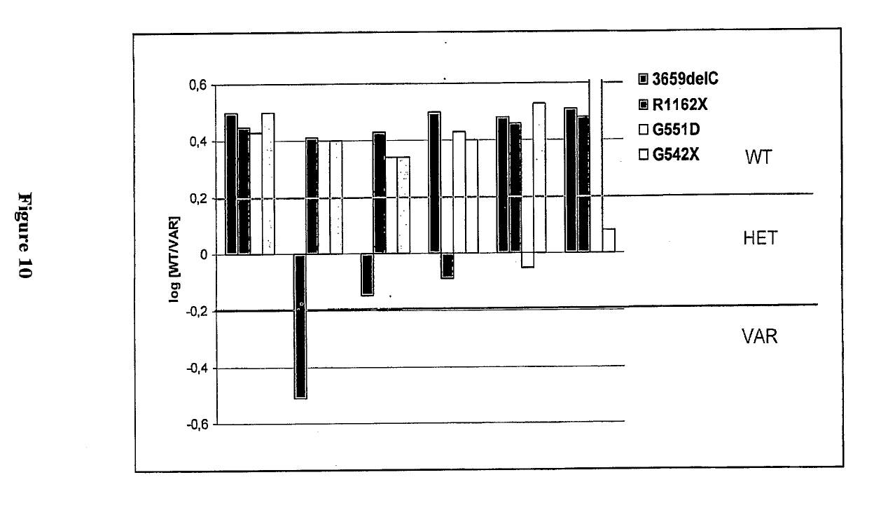

Genotyped DNA samples were obtained from the Coriell Cell Repository and were sonicated to shear the genomic DNA to <1 kb size. Following sonication, 35 cycles of primer extensions were performed in the presence of biotinylated dCTP. Hybridization to the chip surface and simultaneous labeling with Cy5-streptavidin conjugate was completed in 15 minutes. Results obtained using this method are presented on FIG. 10. As little as 50 ng of genomic DNA was sufficient to correctly determine the genotype of the six targets.

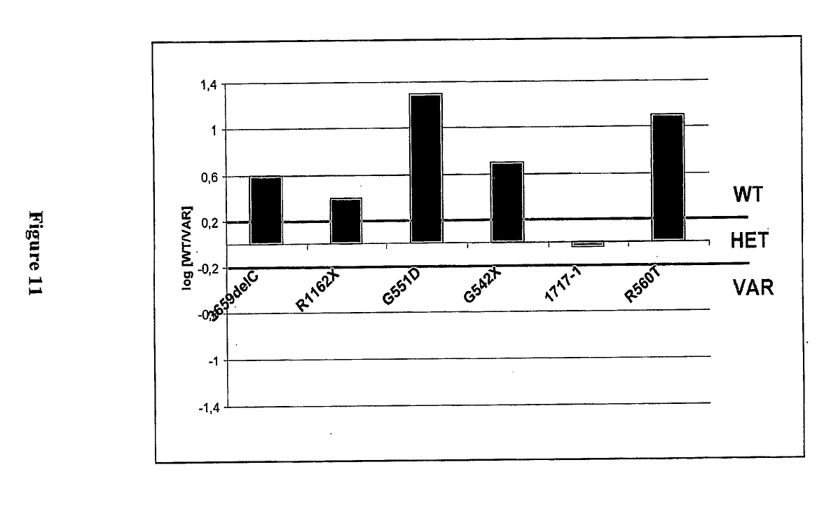

The allele-specific hybridization based format, termed “capture assisted differential hybridization or CADH”, does not use any enzymatic steps and combines a highly selective target capture method using mixtures of immobilized target specific capture probes, with Cy5 labeled ASH probes for discrimination. In this method, genomic DNA samples are sonicated, then incubated with the ASH probes on the chip in a single capture-discrimination step. The feasibility of CADH was demonstrated with ten different specimens. Some of the results obtained using this method are presented in FIG. 11. The CFTR SNPs G551D and R1162X were correctly determined in all cases using 25 μg of genomic DNA input.

Both ASPE and CADH can therefore be employed on PWG to enable SNP detection from genomic samples without target amplification. ASPE needs less sample than CADH, but requires an enzymatic step. Based on these results, it appears that ASPE may be a suitable technology for genotyping in the central lab, whereas CADH may be especially promising with regards to multiplexed SNP analysis using planar waveguides at the point of care.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A method for genotyping one or more single nucleotide polymorphic loci in a nucleic acid sample, the method comprising steps of:
providing a sample comprising nucleic acid molecules of higher biological complexity relative to amplified nucleic acid molecules, the nucleic acid molecules of the sample including a plurality of target regions, each target regions having a single nucleotide polymorphic locus;
combining said sample with at least one set of primers specific for a first single nucleotide polymorphic locus in a first target region;
performing primer extension to obtain extension products; and
identifying the primer extension products obtained, wherein said step of identifying allows the genotype of said one or more single nucleotide polymorphic loci to be established.

2. The method of claim 1, wherein at least two sets of primers are combined with said sample and each set of primers is specific for one particular single nucleotide polymorphic locus in a particular target region.

3. The method of claim 1, wherein the step of providing a sample comprising nucleic acid molecules of higher biological complexity relative to amplified nucleic acid molecules comprises steps of: obtaining a sample of genomic DNA that has not been amplified and fragmenting the genomic DNA.

4. The method of claim 3, wherein the step of fragmenting comprises submitting the genomic DNA to sonication.

5. The method of claim 4, wherein sonication yields genomic DNA fragments of less than 2 kb in size or less than 1 kb in size.

6. The method of claim 1, wherein the at least one set of primers specific for a first single nucleotide polymorphic locus in a first target region comprises a first allele-specific primer and a second allele-specific primer, wherein:
the first allele-specific primer comprises a 3′ portion that hybridizes to a portion of the first target region immediately adjacent to the first single nucleotide polymorphic locus and that has a 3′-terminal nucleotide that is complementary to a non-mutated base at said locus, and a 5′ portion that is complementary to all or part of a first pre-selected nucleic acid sequence which is different from sequences of the nucleic aid molecules of the sample; and
the second allele-specific primer comprises a 3′ portion that hybridizes to a portion of the first target region immediately adjacent to the first single nucleotide polymorphic locus and that has a 3′-terminal nucleotide that is complementary to a mutated base at said locus, and a 5′ portion that is complementary to all or part of a second pre-selected nucleic acid sequence which is different from sequences of the nucleic acid molecules of the sample.

7. The method of claim 6, wherein the at least one set of primers specific for said first single nucleotide polymorphic locus in said first target region further comprises at least one non-extendable oligonucleotide probe, wherein said non-extendable oligonucleotide probe comprises a 5′ portion that is complementary to a portion of said first target region 3′ to the first single nucleotide polymorphic locus and has at least two 3′-terminal nucleotides that are not complementary to the target region.

8. The method of claim 6, wherein the step of performing primer extension to obtain primer extension products comprises using polymerase chain reaction (PCR).

9. The method of claim 8, wherein the step of performing primer extension with PCR is conducted using non-proofreading polymerase enzyme.

10. The method of claim 9, wherein the step of performing primer extension with PCR is conducted using a DNA polymerase which lacks 5′→3′ exonuclease activity or which lacks both 5′→3′ exonuclease activity and 3′→5′ exonuclease activity.

11. The method of claim 9, wherein the step of performing primer extension with PCR comprises extending primers in an allele-specific manner and incorporating nucleoside triphosphates from solution, a plurality of the nucleotides incorporated in the extension products being labeled nucleotides, thereby obtaining labeled primer extension products.

12. The method of claim 11 further comprising steps of: subjecting said labeled primer extension products to hybridization conditions with at least one set of pre-selected nucleic acid sequences, wherein:
the set of pre-selected nucleic acid sequences is associated with the set of primers specific for one first single nucleotide polymorphic locus in a first target sequence and comprises: a first pre-selected nucleic acid sequence which is, at least in part, complementary to the 5′ portion of the first allele-specific primer of said primer set; and a second pre-selected nucleic acid sequence which is, at least in part, complementary to the 5′ portion of the second allele-specific primer of said primer set; and
determining whether hybridization occurs, wherein hybridization to the first pre-selected nucleic acid sequence indicates that the nucleic acid sample contains, at said first single nucleotide polymorphic locus, a nucleotide that is complementary to the 3′-terminal nucleotide of the first allele-specific primer, and wherein hybridization to the second pre-selected nucleic acid sequence indicates that the nucleic acid sample contains, at said first single nucleotide polymorphic locus, a nucleotide that is complementary to the 3′-terminal nucleotide of the second allele-specific primer.

13. The method of claim 12, wherein at least two sets of pre-selected nucleic acid sequences are used and wherein each set of pre-selected nucleic acid sequences is associated with one set of primers specific for one particular single nucleotide polymorphic locus in a particular target region.

14. The method of claim 13, wherein the pre-selected nucleic acid sequences are randomly generated.

15. The method of claim 13, wherein the pre-selected nucleic acid sequences are immobilized on a solid support.

16. The method of claim 15, wherein the solid support comprises an array.

17. The method of claim 15, wherein the solid support comprises a set of beads.

18. The method of claim 12, wherein the first pre-selected nucleic acid sequence is immobilized at a first pre-selected discrete location in an array of immobilized, pre-selected nucleic acid sequences, and wherein said second pre-selected nucleic acid sequence is immobilized at a second pre-selected discrete location in said array.

19. The method of claim 18, wherein the first discrete location is associated with the nucleotide at the first single nucleotide polymorphic locus being a non-mutated base, and wherein the second discrete location is associated with the nucleotide at said locus being a mutated base.

20. The method of claim 12, wherein the first pre-selected nucleic acid sequence is immobilized on a first coded solid support and the second pre-selected nucleic acid sequence is immobilized on a second coded solid support.

21. The method of claim 20, wherein the first coded solid support is associated with the nucleotide at the first single nucleotide polymorphic locus being a non-mutated base, and wherein the second coded solid support is associated with the nucleotide at said locus being a mutated base.

22. The method of claim 15, wherein the step of determining whether hybridization occurs comprises a step of detecting labeled primer extension products hybridized to pre-selected nucleic acid sequences immobilized on a solid support.

23. The method of claim 22, wherein the step of detecting is performed using a photonic, electronic, acoustic, opto-acoustic, electrochemical, electro-optic, mass-spectrometric, enzymatic, chemical, biochemical, physical technique or a combination thereof.

24. The method of claim 22, wherein the step of detecting is performed using a planar waveguide chip technique.

25. A kit for genotyping one or more single nucleotide polymorphic loci in a nucleic acid sample, the kit comprising:
one or more sets of primers, wherein each set of primers is specific for one particular single nucleotide polymorphic locus in a particular target region;
one or more sets of pre-selected nucleic acid sequences, wherein each set of pre-selected nucleic acid sequences is associated with one set of primers; and
instructions for using the kit according to claim 1.

26. The kit of claim 25, wherein a set of primers specific for one particular single nucleotide polymorphic locus in a particular target region comprises a first allele-specific primer and a second allele-specific primer, wherein:
the first allele-specific primer comprises a 3′ portion that hybridizes to a portion of said particular target region immediately adjacent to said particular single nucleotide polymorphic locus and has a 3′-terminal nucleotide that is complementary to a non-mutated base at said locus, and a 5′ portion that is complementary to all or part of a first pre-selected nucleic acid sequence which is different from sequences of the nucleic acid molecules of the sample; and wherein
the second allele-specific primer comprises a 3′ portion that hybridizes to a portion of said particular target region immediately adjacent to said particular single nucleotide polymorphic locus and has a 3′-terminal nucleotide that is complementary to a mutated base at said locus, and a 5′ portion that is complementary to all or part of a second pre-selected nucleic acid sequence which is different from sequences of the nucleic acid molecules of the sample.

27. The kit of claim 26, wherein a set of pre-selected nucleic acid sequences associated with one set of primers specific for one particular single nucleotide polymorphic locus in a particular target region comprises a first pre-selected nucleic acid sequence and a second pre-selected nucleic acid sequence, wherein:
the first pre-selected nucleic acid sequence is, at least in part, complementary to the 5′ portion of the first allele-specific primer of said primer set; and
the second pre-selected nucleic acid sequence is, at least in part, complementary to the 5′ portion of the second allele-specific primer of said primer set.

28. The kit of claim 27, wherein the pre-selected nucleic acid sequences are randomly generated.

29. The kit of claim 27, wherein the pre-selected nucleic acid sequences are immobilized on a solid support.

30. The kit of claim 29, wherein the solid support comprises an array.

31. The kit of claim 29, wherein the solid support comprises a set of beads.

32. The kit of claim 29, wherein the first and second pre-selected nucleic acid sequences of each set of pre-selected nucleic acid sequences are immobilized at a first and second pre-selected discrete locations on an array.

33. The kit of claim 29, wherein the first and second pre-selected nucleic acid sequences of each set of pre-selected nucleic acid sequences are immobilized on a first and second coded solid supports.

34. The kit of claim 26, wherein each set of primers further comprises at least one non-extendable oligonucleotide probe.

35. The kit of claim 27 further comprising a non-proofreading polymerase enzyme.

36. The kit of claim 27 further comprising a DNA polymerase which lacks 5′→3′ exonuclease activity or which lacks both 5′→3′ exonuclease activity and 3′→5′ exonuclease activity.
